(19) United States
(12) Patent Application Publication
(10) Pub. No.: US 2018/0250053 A1
(43) Pub. Date: Sep. 6, 2018

(54) Line Passing Devices and Related Kits and Methods

(71) Applicant: Scott & White Healthcare, Temple, TX (US)

(72) Inventor: Richard SCHULTZ, Temple, TX (US)

(21) Appl. No.: 15/771,808

(22) PCT Filed: Oct. 27, 2016
(86) PCT No.: PCT/US16/59162

§ 371 Date: Apr. 27, 2018

Related U.S. Application Data
(60) Provisional application No. 62/247,084, filed on Oct. 27, 2015.

(51) Int. Cl. A61B 17/88; A61B 17/82; A61B 17/06

(52) U.S. Cl. CPC A61B 17/8861; A61B 2017/00314; A61B 17/06066; A61B 17/82

(57) ABSTRACT

Some of the present line passing devices include an elongated body having a proximal end, a distal end including a tip and an actuatable portion proximal to the tip, and a line passageway sized to receive a line and extending in a direction from the proximal end to the distal end, the line passageway extending through the distal end, a user-operable adjustment member movably coupled to the proximal end, and one or more wires coupled to the knob and to the actuatable portion, where the line passing device is configured such that, in response to movement of the knob, the one or more wires actuate the actuatable portion to angularly displace the distal end relative to the proximal end.

This application claims the benefit of priority to U.S. provisional patent application Ser. No. 62/247,084, filed Oct. 27, 2015, hereby incorporated by reference in its entirety.

### BACKGROUND

### 1. Field of Invention

The present invention relates generally to devices, kits, and methods for use in medical procedures in which a line (e.g., a cable or suture) is passed around and/or through a structure, such as, for example, orthopedic cabling procedures in which a cerclage cable is passed around at least a portion of a bone.

### 2. Description of Related Art

Provided by way of example, in a typical orthopedic cabling or cerclage procedure, a cerclage cable is passed around at least a portion of a bone and is tensioned, whereby the cerclage cable may be used to hold fractured and/or fragmented portions of the bone together (e.g., to facilitate healing of the bone), to fasten device(s), such as bone plate(s), to the bone, and/or the like. Typically, a cerclage cable is passed around a bone using a line passing device.

Such line passing devices typically include a curved section configured to guide a cerclage cable around a bone. For many line passing devices, this curved section is fixed in shape and rigid to resist deformation as the line passing device is maneuvered to and into position around a bone. However, such a fixed, rigid curved section of a line passing device may necessitate a larger incision at an insertion site on a patient, cause increased trauma as the line passing device is maneuvered to and into position around a bone, and/or the like.

Some line passing devices may include a curved section that is adjustable in shape. However, such an adjustable curved section of a line passing device may tend to undesirably deform as the line passing device is maneuvered to and into position around a bone, complicating a procedure, causing increased trauma to a patient, and/or the like. Additionally, many such line passing devices require a clinician to pre-shape the adjustable curved section prior to use, giving rise to problems similar to those caused by line passing devices having fixed, rigid curved sections.

Examples of line passing devices are disclosed in U.S. Pat. No. 5,851,209 and in Pub. Nos. US 2004/0010264 and US 2013/0110134.

### SUMMARY

Some embodiments of the present line passing devices are configured, through an elongated body having a proximal end and a distal end including an actuatable portion that is movable between a first rigid position in which the distal end is axially aligned with the proximal end and a second rigid position in which the distal end is not axially aligned with the proximal end, to, for example, require a relatively small incision for insertion of the line passing device into the patient, reduce trauma to the patient during insertion, and/or resist deformation during insertion and/or positioning of the line passing device around at least a portion of an anatomical structure of the patient, such as, for example, a bone.

Some embodiments of the present line passing devices are configured, through a penetrator having a tip and an actuatable portion proximal to the tip and one or more line retainers coupled to and disposed alongside the penetrator that define a line passageway, to, for example, have enhanced penetrating ability (e.g., by having a solid or non-hollow tip, any one of a wider variety of tip configurations, and/or the like).

Some embodiments of the present line passing devices are configured, through an elongated body having a proximal end and a distal end including an actuatable portion and a knob movably coupled to the proximal end and configured to actuate the actuatable portion to angularly displace the distal end relative to the proximal end, to, for example, allow a clinician to adjust a position of the distal end relative to the proximal end from outside of a patient's body when the distal end is disposed within the patient's body.

Some embodiments of the present line passing devices comprise: an elongated body having a proximal end, a distal end including a tip and an actuatable portion proximal to the tip, and a line passageway sized to receive a line and extending in a direction from the proximal end to the distal end, the line passageway extending through the distal end, a knob movably coupled to the proximal end, and one or more wires coupled to the knob and the actuatable portion, where the line passing device is configured such that, in response to movement of the knob, the one or more wires actuate the actuatable portion to angularly displace the distal end relative to the proximal end. In some embodiments, the body includes a penetrator having the tip and one or more line retainers coupled to and disposed alongside the penetrator, the one or more line retainers defining at least a portion of the line passageway.

In some embodiments, the one or more line retainers includes an elongated cannula. Some embodiments comprise a line (e.g., a cable or a suture) at least partially disposed within the line passageway.

In some embodiments, the body includes one or more interior passageways, each configured to receive at least one of the one or more wires. In some embodiments, the body includes a rigid shaft disposed between the proximal end and the actuatable portion. In some embodiments, the proximal end of the body includes a handle. In some embodiments, the tip is tapered. In some embodiments, the tip is curved. In some embodiments, the tip is serrated.

In some embodiments, the line passing device is configured such that, in response to movement of the knob, the one or more wires actuate the actuatable portion between a first rigid position in which the distal end is axially aligned with the proximal end and a second rigid position in which the distal end is not axially aligned with the proximal end. In some embodiments, the actuatable portion is actuatable such that the distal end is angularly displaced relative to the proximal end about a first axis and about a second axis that is perpendicular to the first axis. In some embodiments, the actuatable portion extends along a majority of a length of the body.

In some embodiments, the actuatable portion comprises a plurality of segments, including at least two adjacent segments, where the actuatable portion is configured such that, in response to movement of the knob, the at least two adjacent segments angularly displace relative to one another. In some embodiments, the at least two adjacent segments each define a first mating surface and a second mating surface, the second mating surface of at least one of the at least two adjacent segments is angularly disposed relative to the first mating surface of the at least one of the at least two adjacent segments, and, in response to movement of the knob, the at least two adjacent segments are movable relative to one another between a first position in which the at least two adjacent segments are in contact with one another along the first mating surface and a second position in which the at least two adjacent segments are in contact with one another along the second mating surface.

In some embodiments, at least one of the plurality of segments is unitary with at least one other of the plurality of segments. Some embodiments comprise a flexible shaft disposed through at least two of the plurality of segments. In some embodiments, at least one of the plurality of segments includes at least one of the one or more line retainers.

Some embodiments of the present line passing devices comprise: an elongated body extending between a proximal end and a distal end, the elongated body including a shaft, an actuatable portion coupled to and distal to the shaft, a tip coupled to and distal to the actuatable portion, and a line passageway sized to receive a line and extending in a direction from the proximal end to the distal end, the line passageway extending through the distal end, and an actuation mechanism comprising first and second carrier members, each carrier member coupled to the body such that the carrier member is translatable relative to the body, one or more wires coupled between the first carrier member and the actuatable portion such that proximal translation of the first carrier member relative to the body actuates the actuatable portion to angularly displace the tip relative to the shaft in a first direction, and one or more wires coupled between the second carrier member and the actuatable portion such that proximal translation of the second carrier member relative to the body actuates the actuatable portion to angularly displace the tip relative to the shaft in a second direction that is opposite to the first direction.

In some embodiments, the actuation mechanism comprises a shaft coupled to the body, the shaft having first and second threaded portions, each threaded portion being rotatable relative to the body, the first carrier member is threadably coupled to the first threaded portion such that rotation of the first threaded portion relative to the body translates the first carrier member relative to the body, and the second carrier member is threadably coupled to the second threaded portion such that rotation of the second threaded portion relative to the body translates the second carrier member relative to the body. In some embodiments, the first threaded portion is rotatable relative to the body independently of rotation of the second threaded portion relative to the body. In some embodiments, the shaft of the actuation mechanism includes an outer spindle defining an interior channel, the outer spindle including the first threaded portion, and an inner spindle rotatably disposed within and extending from the interior channel of the outer spindle, wherein a portion of the inner spindle that is disposed outside of the interior channel includes the second threaded portion.

In some embodiments, the actuation mechanism comprises a knob coupled to the shaft of the actuation mechanism such that rotation of the knob relative to the body rotates at least one of the first threaded portion and the second threaded portion relative to the body. In some embodiments, the actuation mechanism comprises a first knob coupled to the shaft of the actuation mechanism such that rotation of the first knob relative to the body rotates the first threaded portion relative to the body, and a second knob coupled to the shaft of the actuation mechanism such that rotation of the second knob relative to the body rotates the second threaded portion relative to the body. In some embodiments, the first knob is movable relative to the second knob between a first position in which the first knob is engaged with the second knob such that rotation of the second knob relative to the body rotates the first knob relative to the body, and a second position in which the first knob is disengaged with the second knob such that the second knob is rotatable relative to the body independently of rotation of the first knob relative to the body.

In some embodiments, the actuatable portion includes a plurality of segments, each having a proximal face and/or a distal face that defines a first mating surface and a second mating surface, for at least one of the faces of at least one of the segments, the second mating surface is angularly disposed relative to the first mating surface, and the actuatable portion is actuatable between a first position in which adjacent ones of the segments are in contact with one another along their first mating surfaces, and a second position in which adjacent ones of the segments are in contact with one another along their second mating surfaces. In some embodiments, when the actuatable portion is in the first position, the tip is axially aligned with the shaft of the body, and, when the actuatable portion is in the second position, the tip is not axially aligned with the shaft of the body.

Some embodiments of the present line passing devices comprise: an elongated body extending between a proximal end and a distal end, the elongated body including a shaft, an actuatable portion coupled to and distal to the shaft, the actuatable portion including a plurality of segments, each having a proximal face and/or a distal face that defines a first mating surface and a second mating surface, wherein, for at least one of the faces of at least one of the segments, the second mating surface is angularly disposed relative to the first mating surface, a tip coupled to and distal to the actuatable portion, and a line passageway sized to receive a line and extending in a direction from the proximal end to the distal end, the line passageway extending through the distal end, and an actuation mechanism comprising one or more wires coupled to the actuatable portion, the one or more wires configured to angularly displace the tip relative to the shaft by actuating the actuatable portion between a first position in which adjacent ones of the segments are in contact with one another along their first mating surfaces, and a second position in which adjacent ones of the segments are in contact with one another along their second mating surfaces.

In some embodiments, when the actuatable portion is in the first position, adjacent ones of the segments are not in contact with one another along their second mating surfaces, and, when the actuatable portion is in the second position, adjacent ones of the segments are not in contact with one another along their first mating surfaces. In some embodiments, when the actuatable portion is in the first position, the tip is axially aligned with the shaft, and, when the actuatable portion is in the second position, the tip is not axially aligned with the shaft.

In some embodiments, for each of the segments, at least one of the faces includes an arcuate bearing surface disposed between the first mating surface and the second mating surface, and the arcuate bearing surface is configured to contact a corresponding arcuate bearing surface of an adjacent one of the segments as the actuatable portion is actuated between the first position and the second position. In some embodiments, for at least one of the segments, the arcuate bearing surface(s) are conical.

In some embodiments, the actuation mechanism comprises a first carrier member coupled to the body such that the first carrier member is translatable relative to the body, and at least one of the one or more wires is coupled between the first carrier member and the actuatable portion such that translation of the first carrier member relative to the body actuates the actuatable portion to angularly displace the tip relative to the shaft. In some embodiments, the actuation mechanism comprises a second carrier member coupled to the body such that the second carrier member is translatable relative to the body, and at least one of the one or more wires is coupled between the second carrier member and the actuatable portion such that translation of the second carrier member relative to the body actuates the actuatable portion to angularly displace the tip relative to the shaft. In some embodiments, proximal translation of the first carrier member relative to the body angularly displaces the tip relative to the shaft in a first direction, and proximal translation of the second carrier member relative to the body angularly displaces the tip relative to the shaft in a second direction that is opposite to the first direction.

In some embodiments, the body includes a handle coupled to and proximal to the shaft of the body. In some embodiments, the tip defines an opening in communication with the line passageway, the opening being defined through a side of the tip. In some embodiments, the shaft of the body defines an opening in communication with the line passageway, the opening being defined through a side of the shaft.

Some embodiments of the present line passing devices, while otherwise similar to embodiment(s) disclosed above, do not include a line passageway, or at least a line passageway that extends within and/or through the shaft, actuatable portion, and/or tip. In some embodiments of the present line passing devices, the distal end of the body can be configured to be coupled to a line. In some embodiments, the distal end of the body defines an opening configured to receive the line. In some embodiments, the line includes a fitting coupled to an end of the line, the opening includes a first portion having a first width and a second portion in communication with the first portion, the second portion having a second width that is smaller than the first width, and the line is configured to be coupled to the distal end of the body by inserting the fitting through the first portion of the opening and moving the line relative to the opening such that the line is disposed within the second portion of the opening.

Some embodiments of the present kits comprise: a line passing device comprising an elongated body having a proximal end, a distal end including a tip and an actuatable portion proximal to the tip, and a line passageway sized to receive a line and extending in a direction from the proximal end to the distal end, the line passageway extending through the distal end, a knob movably coupled to the proximal end, and one or more wires coupled to the knob and to the actuatable portion, where the line passing device is configured such that, in response to movement of the knob, the one or more wires actuate the actuatable portion to angularly displace the distal end relative to the proximal end.

In some embodiments, the body includes a penetrator having the tip and one or more line retainers coupled to and disposed alongside the penetrator, the one or more line retainers defining at least a portion of the line passageway. In some embodiments, the line passing device is configured such that, in response to movement of the knob, the one or more wires actuate the actuatable portion between a first rigid position in which the distal end is axially aligned with the proximal end and a second rigid position in which the distal end is not aligned with the proximal end.

Some embodiments comprise a cerclage cable sized to be received within the line passageway. In some embodiments, the cerclage cable is at least partially disposed within the line passageway. Some embodiments comprise a suture sized to be received within the line passageway. In some embodiments, the suture is at least partially disposed within the line passageway. Some embodiments comprise a tunneler.

Some embodiments of the present methods for performing a cerclage comprise: inserting a distal end of an elongated body into an insertion site on a patient, the body including a proximal end and a line passageway extending in a direction from the proximal end to the distal end, where the distal end comprises an actuatable portion, actuating the actuatable portion to angularly displace the distal end relative to the proximal end such that at least a portion of the body at least partially surrounds a bone of the patient, and passing a cerclage cable out of the line passageway and at least partially around the bone.

Some embodiments comprise actuating the actuatable portion to angularly displace the distal end relative to the proximal end toward a position in which the distal end is axially aligned with the proximal end and removing the body from the insertion site. Some embodiments comprise tensioning the cerclage cable Some embodiments comprise inserting a tunneler into the insertion site on the patient.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those one or more elements. Likewise, a method that “comprises,” “has,” or “includes,” one or more steps possesses those one or more steps, but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments are described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. In the figures, reference numerals having the same number but different suffixes (e.g., a, b, c, d, or the like) may be used to refer to similar features or features with similar functionality. The figures are drawn to scale (unless otherwise noted), meaning the sizes of the depicted elements are accurate relative to each other for at least the embodiment depicted in the figures.

FIG. 1A is a top view of a first embodiment of the present line passing devices.

FIG. 1B is a top view of a portion of the embodiment of FIG. 1A.

FIGS. 1C-1E are bottom, side, and front views, respectively, of the embodiment of FIG. 1A.

FIG. 1F is a cross-sectional side view of the embodiment of FIG. 1A.

FIG. 1G is a side view of the embodiment of FIG. 1A, shown with an actuatable portion in a first position.

FIG. 1H is a side view of the embodiment of FIG. 1A, shown with the actuatable portion in a second position.

FIGS. 2A and 2B each depict a tip, which may be suitable for use in some embodiments of the present line passing devices.

FIG. 3A is a cross-sectional side view of the embodiment of FIG. 1A, shown with the actuatable portion in the first position.

FIG. 3B is a cross-sectional side view of the embodiment of FIG. 1A, shown with the actuatable portion in the second position.

FIGS. 4A-4C each depict a partially cross-sectional view of a segment, which may be suitable for use in some embodiments of the present line passing devices.

FIG. 5 is a cross-sectional side view of the embodiment of FIG. 1A, showing an exemplary actuation mechanism.

FIG. 6 is a top view of a second embodiment of the present line passing devices.

FIG. 7 is a top view of a third embodiment of the present line passing devices.

FIG. 8 is a side view of a cerclage tunneler, which may be suitable for use with some embodiments of the present line passing devices.

FIG. 9 is a perspective view of a fourth embodiment of the present line passing devices.

FIGS. 10-12 are side, top, and bottom views, respectively, of the embodiment of FIG. 9.

FIGS. 13 and 14 are front and back views, respectively, of the embodiment of FIG. 9.

FIG. 15 is a cross-sectional side view of the embodiment of FIG. 9.

FIG. 16 is a cross-sectional side view of the embodiment of FIG. 9, shown with an actuatable portion in a second position.

FIG. 17 is a perspective view of a segment, which may be suitable for use in some embodiments of the present line passing devices.

FIGS. 18-20 are back, front, and side views, respectively, of the segment of FIG. 17.

FIG. 21 is a cross-sectional side view of the segment of FIG. 17.

FIGS. 22 and 23 are top and bottom views, respectively, of the segment of FIG. 17.

FIG. 24 is a cross-sectional side view of the embodiment of FIG. 9, showing an exemplary actuation mechanism.

FIGS. 25A and 25B are cross-sectional side views of knobs that may be suitable for use as user-operable adjustment members in some embodiments of the present line passing devices, shown in first and second positions, respectively.

FIG. 26 is a side view of a fifth embodiment of the present line passing devices.

FIG. 27 depicts a line that may be suitable for use with some embodiments of the present line passing devices.

### DETAILED DESCRIPTION OF ILLUSTRATIVE EMBODIMENTS

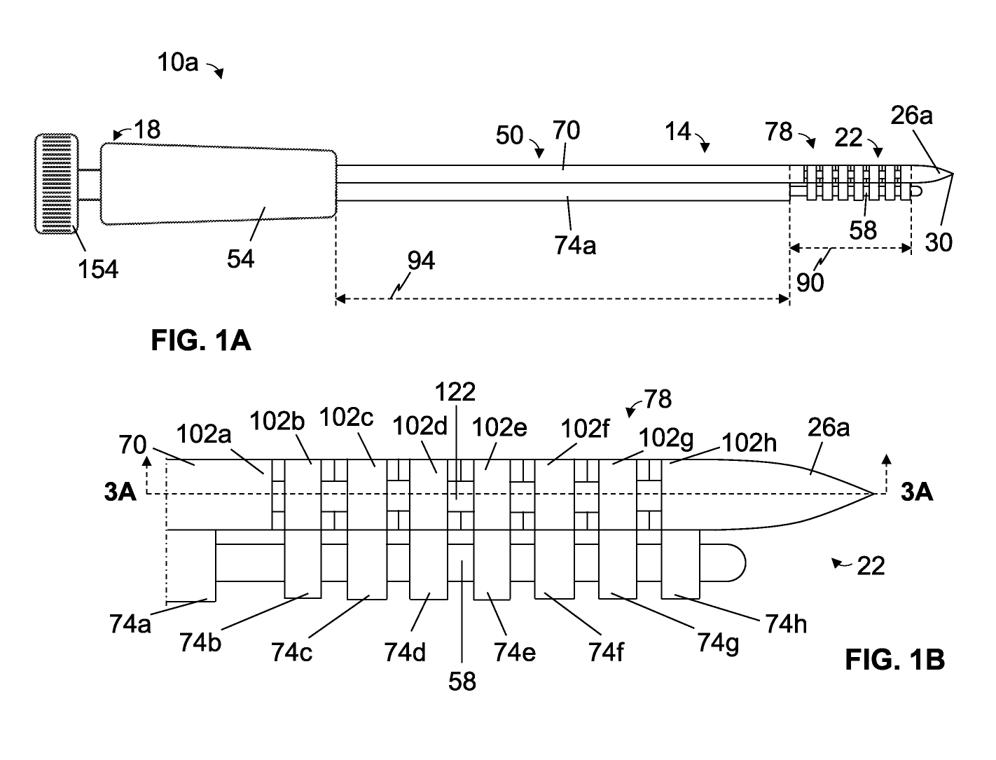

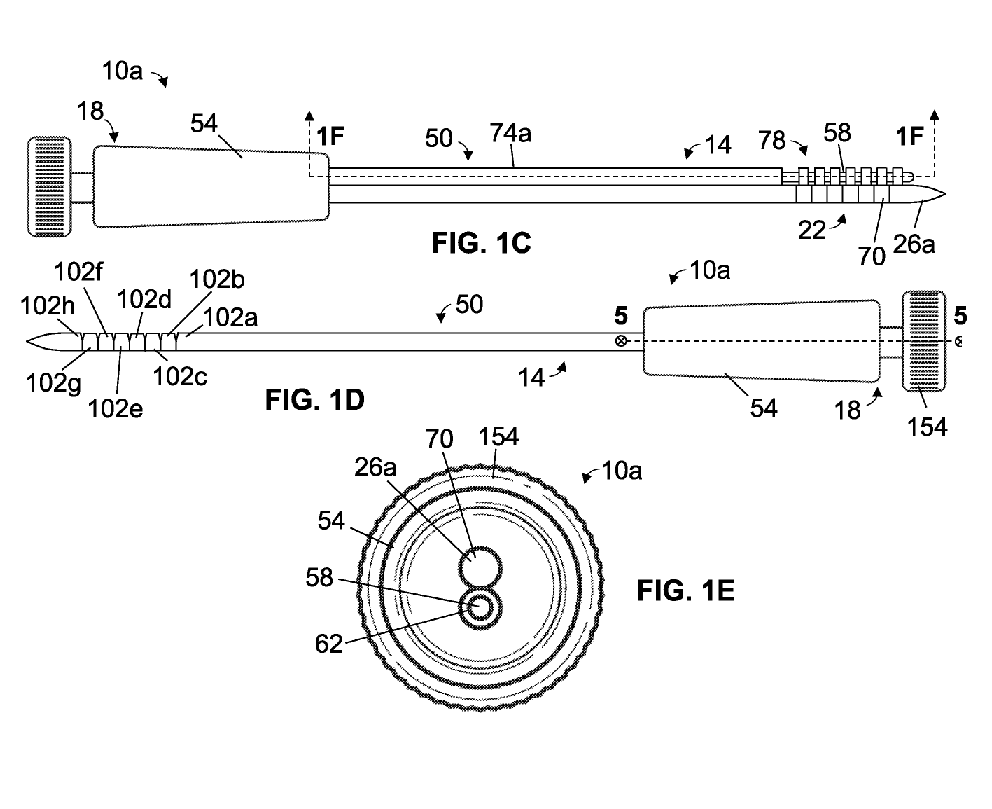

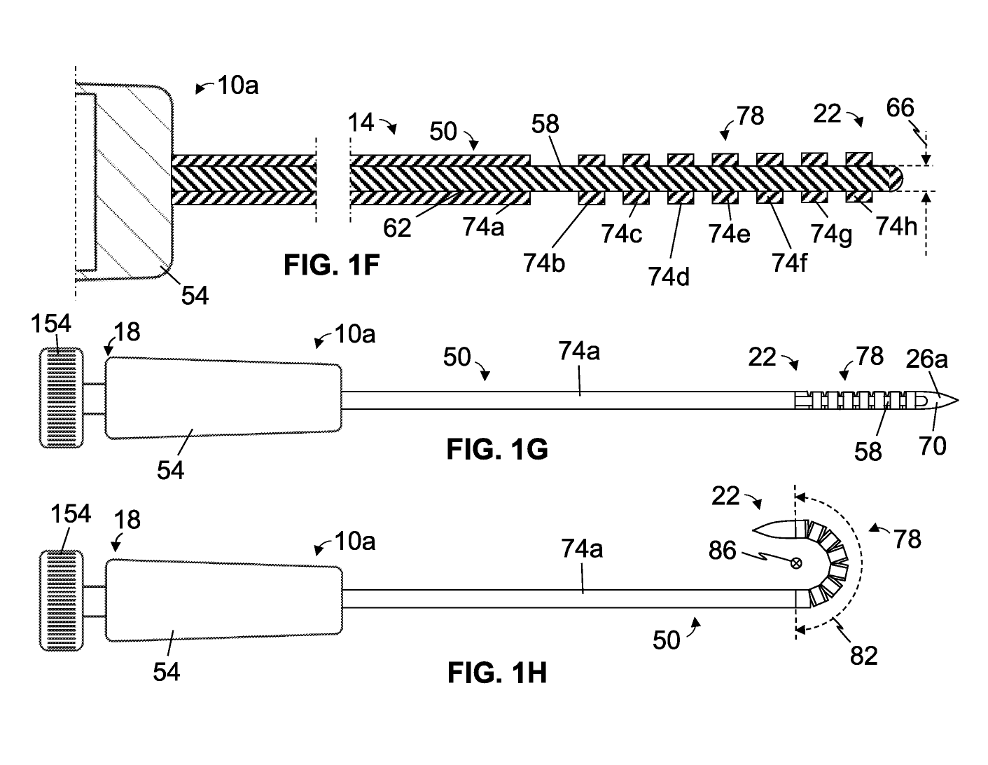

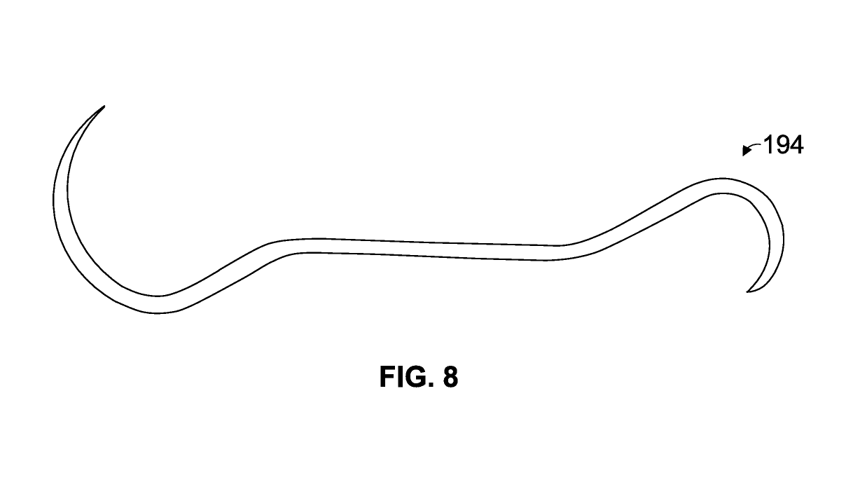

Referring now to the figures, and more particularly to FIGS. 1A-1H, shown therein and designated by the reference numeral 10a is a first embodiment of the present line passing devices. In the embodiment shown, device 10a comprises an elongated body 14 extending between a proximal end 18 and a distal end 22. As shown, distal end 22 of elongated body 14 includes a tip (e.g., 26a). Bodies (e.g., 14) of the present line passing devices may comprise any suitable material, such as, for example, a metal (e.g., stainless steel, aluminum, titanium, and/or the like), plastic, composite material, and/or the like. In this embodiment, tip 26a is configured to facilitate insertion of distal end 22 into and/or through an insertion site on a patient, tissue beneath the insertion site (e.g., muscle, connective tissue, and/or the like, such as that adjacent to a bone), and/or the like. For example, in the depicted embodiment, tip 26a tapers to a point 30. In the embodiment shown, tip 26a is generally straight; for example, point 30 is substantially centered on a longitudinal axis of the tip. However, other embodiments of the present line passing devices may comprise any suitable tip (e.g., 26a), whether straight or curved and whether sharp or rounded (e.g., for use in some embodiments that are configured to be used in conjunction with a separate cerclage tunneler, such as tunneler 194 of FIG. 8).

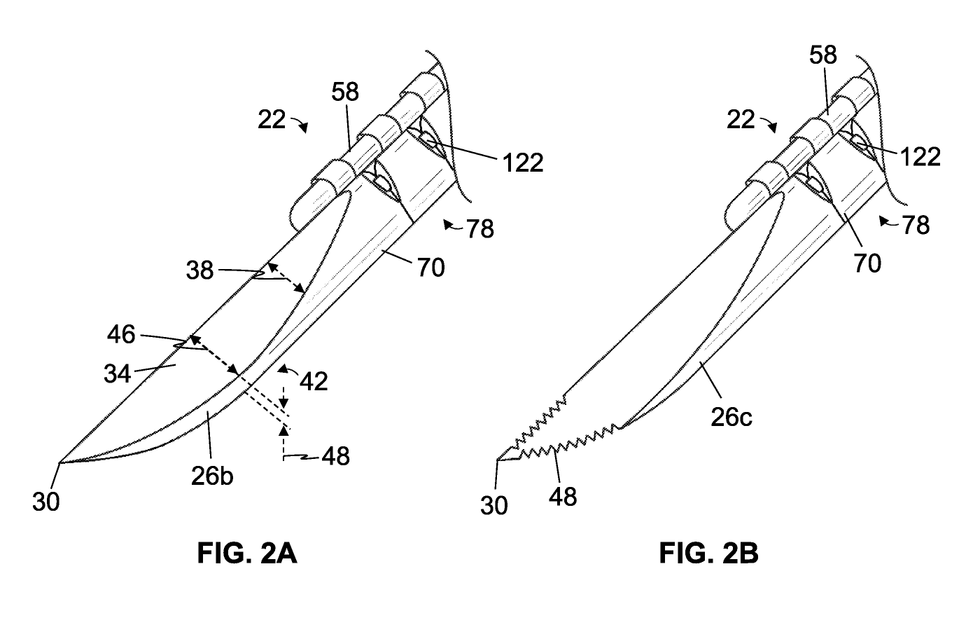

For example, FIGS. 2A and 2B each depict a tip (26b and 26c, respectively), which may be suitable for use in some embodiments of the present line passing devices. As shown in FIG. 2A, tip 26b is generally curved, including a concave surface 34. In this embodiment, concave surface 34 has a width 38 that tapers in a distal direction and/or a proximal direction. In the embodiment shown, a portion 42 of tip 26b includes a non-circular cross section, which may have a width 46 that is larger than a corresponding thickness 48. Through such feature(s) and others, tip 26b may resemble a periosteal elevator. FIG. 2B depicts tip 26c, which is substantially similar to tip 26b, with the primary exception that distal edge(s) of tip 26c define serrations or ridges 48. In these ways and others, tips 26b and/or 26c may facilitate passage of distal end 22 through and/or past tissue adjacent to a bone of a patient (e.g., by facilitating separation between the bone and adjacent tissue).

In this embodiment, device 10a, and more particularly, body 14, includes a (e.g., rigid) shaft 50 disposed between proximal end 18 and distal end 22, and more particularly, between proximal end 18 and actuatable portion 78 of the distal end. In the depicted embodiment, at least a section of shaft 50 includes both a portion of a penetrator 70, which may be rigid, and a portion of one or more line retainers (e.g., 74a-74h) (each described in more detail below), such as a portion of elongated cannula 74a, which may or may not be rigid, that are disposed alongside one another. As a result, in some embodiments, the section of the shaft comprises or resembles a double-barrel construction.

In the embodiment shown, body 14 comprises a handle 54 coupled to and proximal to shaft 50 of the body. Some embodiments may include a handle (e.g., 54) that is contoured, textured, and/or the like and/or that includes gripping features (e.g., flange(s), guard(s), and/or the like) to, for example, mitigate inadvertent slippage of the handle within a clinician's hand during use of the device. Through these feature(s) and others, a clinician using an embodiment of the present line passing devices (e.g., 10a) may be facilitated in insertion of a distal end (e.g., 22) into and/or through an insertion site on a patient, tissue beneath the insertion site (e.g., muscle, connective tissue, and/or the like, such as that adjacent to bone), and/or the like (e.g., via leverage provided by a shaft 50 and/or a handle 54).

In this embodiment, device 10a is configured to insert a line (e.g., 58) into a patient, such as, for example, a cerclage cable or a suture. For example, in the depicted embodiment, body 14 (e.g., one or more line retainers 74a-74h, in this embodiment) defines a line passageway 62 sized to receive a line (e.g., 58) (FIG. 1F). Lines (e.g., 58) of, or suitable for use with, the present line passing devices may include any suitable line having any suitable dimensions, such as, for example, a cable having a diameter greater than or equal to any one of, or between any two of: 0.25, 0.50, 0.75, 1.00, 1.25, 1.50, 1.75, 2.00, 2.25, 2.50, 2.75, or 3.00 millimeters (mm), a suture having a diameter greater than or equal to any one of, or between any two of: 0.010, 0.020, 0.030, 0.050, 0.075, 0.100, 0.150, 0.200, 0.250, 0.300, 0.350, 0.400, 0.500, 0.600, 0.700, or 0.800 mm, or the like. In the embodiment shown, at least a portion of line passageway 62 (e.g., elongated cannula 74a) has a transverse dimension 66 that substantially matches, but is slightly larger than, a diameter of a line (e.g., 58) receivable within the line passageway. As a result, the line may be closely received by, yet slidable within, the at least a portion of the line passageway, which may mitigate clogging of the at least a portion of the line passageway with tissue during use of device 10a.

In this embodiment, line passageway 62 extends in a direction from proximal end 18 of body 14 to distal end 22 of the body. In the embodiment shown, line passageway 62 extends through distal end 22. No portion of device 10a, other than line 58, if present, blocks access to the line passageway from the distal end of the device such that the line may be passed into and out of the line passageway from the distal end of the device. As shown, line passageway 62 may not extend proximally through device 10a. In the depicted embodiment, line passageway 62 is defined by body 14 such that the line passageway is exterior to and positioned alongside penetrator 70. For example, in the embodiment shown, penetrator 70 includes tip 26a, and one or more line retainers (e.g., 74a-74h) that are coupled to and disposed alongside the penetrator define at least a portion of line passageway 62. In this embodiment, line passageway 62, being defined by elements that may be spaced apart from one another (e.g., one or more line retainers 74a-74h), may be open to an exterior of the device at one or more locations along a length of the line passageway. In some embodiments (e.g., 10a), a line (e.g., 58), such as a cable or suture, may be at least partially disposed within (e.g., pre-loaded into) a line passageway (e.g., 62).

In this embodiment, body 14 comprises an actuatable portion 78 configured to actuate to angularly displace distal end 22 of the body relative to proximal end 18 of the body (FIG. 1H) (e.g., in a direction along arrow 82). More particularly, in the embodiment shown, actuatable portion 78 is coupled to and distal to shaft 50, and tip 26a is coupled to and distal to the actuatable portion such that actuation of the actuatable portion angularly displaces the tip relative to the shaft. In the depicted embodiment, actuatable portion 78 is configured to angularly displace distal end 22 relative to proximal end 18 substantially about a (e.g., single) axis 86; however, in other embodiments, an actuatable portion (e.g., 78) may be configured to angularly displace a distal end (e.g., 22) of a body (e.g., 14) relative to a proximal end (e.g., 18) of the body about a first axis (e.g., 86) and about a second axis that is perpendicular to the first axis (e.g., an example of which is described below). In the depicted embodiment, actuatable portion 78 spans a distance 90 along body 14 that is less than 50% of a length 94 of shaft 50 (e.g., less than 20% of the length); however, in other embodiments, an actuatable portion (e.g., 78) may span a distance (e.g., 90) along a body (e.g., 14) that is 50% or more (e.g., a majority of) of length (e.g., 94) of the shaft.

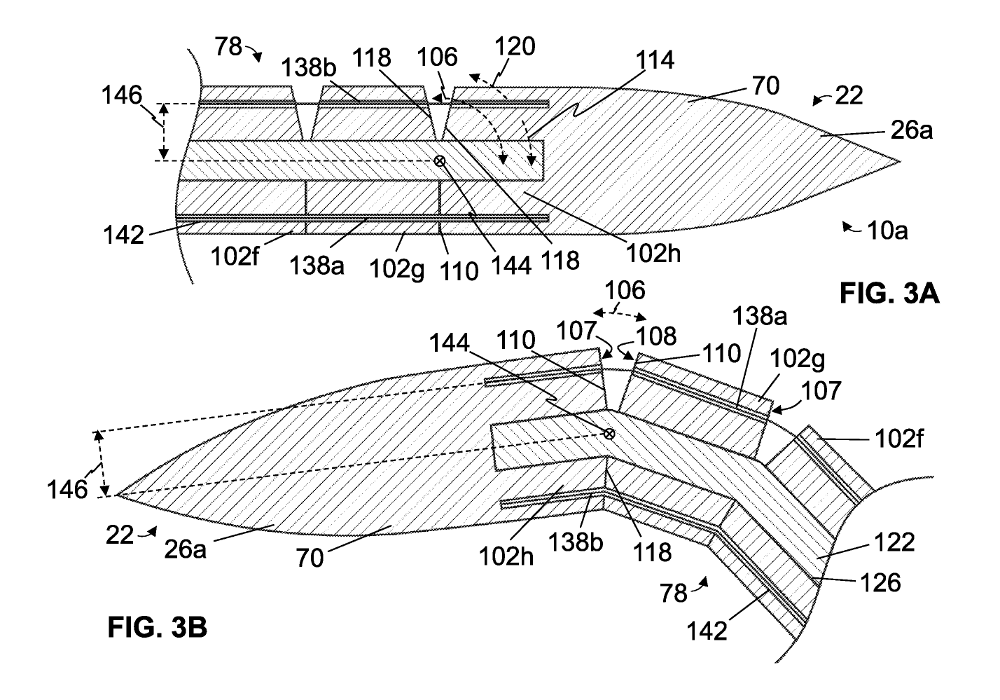

FIGS. 3A and 3B are cross-sectional side views of device 10a, showing a portion of actuatable portion 78 in a first position and a second position, respectively. Actuatable portion 78 is provided only by way of example because the present line passing devices may include any suitable actuatable portion (e.g., 78) that is configured to angularly displace a distal end (e.g., 22) of a body (e.g., 14) relative to a proximal end (e.g., 18) of the body. In the depicted embodiment, actuatable portion 78 comprises a plurality of segments (e.g., 102a-102h), where adjacent segments are configured to angularly displace relative to one another during actuation. For example, in the embodiment shown, during actuation of actuatable portion 78 between the first position (FIG. 3A) and the second position (FIG. 3B), segment 102h (which, in this embodiment, is unitary or integrally formed with tip 26a) angularly displaces relative to segment 102g in a direction along arrow 106 (e.g., about a pivoting axis 144). As shown, device 10a includes eight (8) segments; however, other embodiments of the present line passing devices may include actuatable portions (e.g., 78) having any suitable number of segments (e.g., 2, 3, 4, 5, 6, 7, 8, 9, 10, or more segments).

In this embodiment, adjacent segments are configured to physically limit angular displacement between the adjacent segments. For example, each of segments 102a-102h has a proximal face 107 and/or a distal face 108, each defining a first mating surface 110 and a second mating surface 118. To illustrate, in the depicted embodiment, segment 102b includes a proximal face 107 and a distal face 108, each having a first mating surface 110 and a second mating surface 118, segment 102a includes a distal face 108 having a first mating surface 110 and a second mating surface 118 (e.g., segment 102a does not include a proximal face 107 due to the segment being unitary with shaft 50), and segment 102h includes a proximal face 107 having a first mating surface 110 and a second mating surface 118 (e.g., segment 102h does not include a distal face 108 due to the segment being unitary with tip 26a). In the embodiment shown, for at least one of proximal face 107 and distal face 108 of at least one of segments 102a-102h, second mating surface 118 is angularly disposed relative to first mating surface 110. In some embodiments, at least one segment (e.g., 102a-102h) includes a proximal face (e.g., 107) and/or a distal face (e.g., 108) that defines a first mating surface (e.g., 110) and a second mating surface (e.g., 118) that is not angularly disposed relative to the first mating surface (e.g., the first and second mating surfaces can be co-planar).

In the depicted embodiment, when adjacent ones of segments 102a-102h are at a maximum angular displacement relative to one another in a first direction 114, the adjacent segments are in contact with one another along their first mating surfaces 110 such that, for example, the first mating surfaces are parallel with one another, as shown in FIG. 3A. In the embodiment shown, when the adjacent segments are at the maximum angular displacement relative to one another in the first direction, the adjacent segments are not in contact with one another along their second mating surfaces 118. Similarly, in this embodiment, when adjacent ones of segments 102a-102h are at a maximum angular displacement relative to one another in a second direction 120, the adjacent segments are in contact with one another along their second mating surfaces 118 such that, for example, the second mating surfaces are parallel with one another, as shown in FIG. 3B. In the depicted embodiment, when the adjacent segments are at the maximum angular displacement relative to one another in the second direction, the adjacent segments are not in contact with one another along their first mating surfaces 114. As will be understood, at least by varying the angle between a first mating surface 110 and a second mating surface 118 of a segment, the range of permitted angular displacement, maximum angular displacement in first direction 114, and/or maximum angular displacement in second direction 120 of the segment relative to an adjacent segment may be varied.

While, as described above, actuatable portion 78 is configured to angularly displace distal end 22 relative to proximal end 18 substantially about a (e.g., single) axis 86, as will be understood, in other embodiments that are otherwise the same as or similar to device 10a, a segment may be rotated relative to other(s) of the segments about a longitudinal axis of an actuatable portion (e.g., 78), thereby allowing for multi-axis (e.g., three-dimensional) angular displacements of a distal end (e.g., 22) relative to a proximal end (e.g., 18).

As shown, when each of the plurality of segments is at a maximum angular displacement in first direction 114 relative to each adjacent segment such that each first mating surface 110 of each segment is in contact with a first mating surface 110 of an adjacent segment, actuatable portion 78 may assume a (e.g., rigid, stable) first position (FIG. 3A). In the depicted embodiment, when actuatable portion 78 is in the first position, tip 26a may be substantially axially aligned with other portion(s) of body 14, such as, for example, shaft 50. Thus, when actuatable portion 78 is in the first position, forces exerted by a clinician on device 10a may be transmitted to tip 26a via the first mating surfaces such as, for example, to facilitate insertion of distal end 22 into and/or through an insertion site on a patient, tissue beneath the insertion site (e.g., muscle, connective tissue, and/or the like, such as that adjacent to a bone), and/or the like.

Similarly, in the depicted embodiment, when each of the plurality of segments is at a maximum angular displacement in second direction 120 relative to each adjacent segment such each second mating surface 118 of each segment is in contact with a second mating surface 118 of an adjacent segment, actuatable portion 78 may assume a second (e.g., rigid, stable) position (FIG. 3B). In the depicted embodiment, when actuatable portion 78 is in the second position, tip 26a may not be axially aligned with other portion(s) of body 14, such as, for example, shaft 50. For example, when actuatable portion 78 is in the second position, tip 26a may be angularly disposed relative to shaft 50. Thus, when actuatable portion 78 is in the second position, forces exerted by a clinician on device 10a may be transmitted to tip 26a via the second mating surfaces to, for example, facilitate passage of distal end 22 through and/or past tissue adjacent to a bone of a patient.

Some embodiments of the present devices can include an actuatable portion (e.g., 78) configured such that, when portion(s) of the actuatable portion are in a rigid, stable position (e.g., adjacent segments of the portion(s) are in contact with one another along their first mating surfaces 110 or second mating surfaces 118), other portion(s) of the actuatable portion are flexible (e.g., adjacent segments of the portion(s) are not in contact with one another along their first mating surfaces 110 or second mating surfaces 118). Some embodiments of the present devices can include a portion (e.g., of shaft 50) that remains flexible notwithstanding the position of an actuatable portion (e.g., 78) of the device.

In the embodiment shown, each of the plurality of segments is separate from or non-unitary with other(s) of the segments; thus, no segment is formed from a piece of material that forms any other(s) of the segments. However, in other embodiments, at least one of a plurality of segments may be unitary or integrally formed with at least one other of the segments; for example, at least one pair of adjacent segments may be integrally connected to one another, for example, via a living hinge, which may be defined along a pivoting axis 144 between the adjacent segments. In the depicted embodiment, device 10a comprises a flexible shaft 122 at least partially disposed through at least two of the plurality of segments (e.g., via an opening 126 defined into and/or through each of the segments), which may promote retention of the segments, relative alignment between the segments, rigidity of actuatable portion 78, and/or the like.

Segments 102a-102h are provided solely by way of example, as other embodiments of the present devices can comprise an actuatable portion (e.g., 78) including segments that have any suitable shape(s) and dimension(s), which may be selected based on desired rigid, stable position(s) of the actuatable portion.

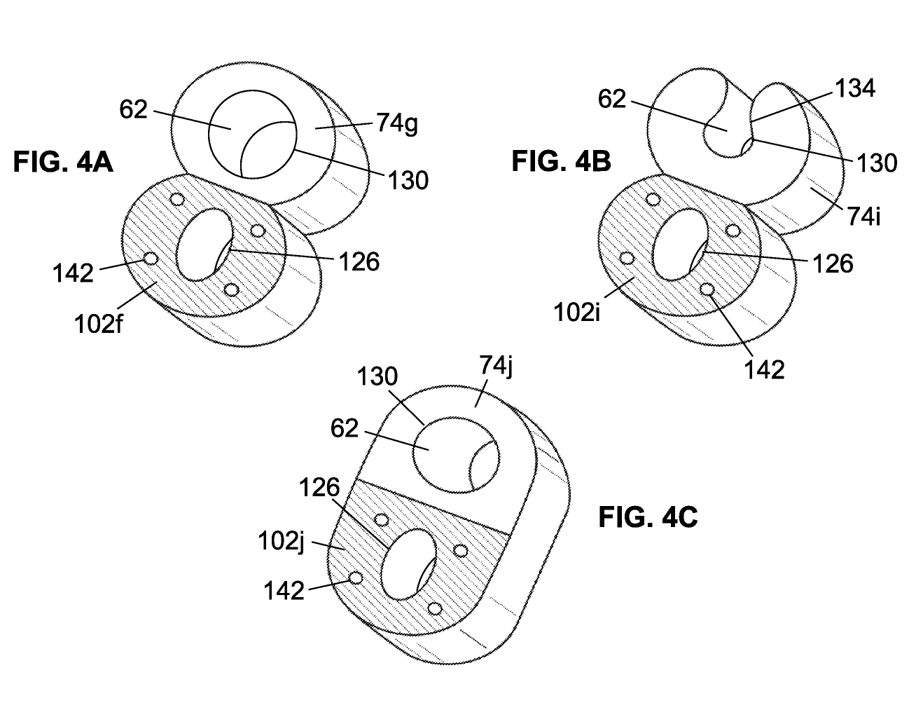

In the depicted embodiment, actuatable portion 78 includes a portion of penetrator 70 (such as portions of the plurality of segments) as well as one or more of the one or more line retainers (FIG. 1B). For example, in this embodiment, actuatable portion 78 includes line retainers 74b-74h, which are spaced apart from one another in a direction along body 14 between proximal end 18 and distal end 22, such as, for example, to facilitate actuation of actuatable portion 78. For further example, and referring additionally to FIG. 4A, in the embodiment shown, at least one of the one or more line retainers (e.g., 74g) is coupled to (e.g., unitary and/or integrally formed with) at least one of the plurality of segments (e.g., 1020. In this embodiment, line retainer 74g includes a generally circular opening 130 having a closed perimeter; however, one or more line retainers of the present line passing devices may comprise any suitable structure that is capable of retaining a line, such as, for example, including a slot 134 in communication with an opening 130 (e.g., line retainer 74i of FIG. 4B), including a non-circular opening (e.g., elliptical opening 130 of line retainer 74j of FIG. 4C, which may mitigate binding between the opening and a line, for example, during actuation of an actuatable portion 78 including line retainer 74j), and/or the like.

In the depicted embodiment, actuatable portion 78 is cable- or wire-actuated. Device 10a includes one or more wires (e.g., four wires, including wires 138a and 138b, which are discussed by way of example) coupled to and configured to actuate actuatable portion 78. For example, in this embodiment, wire 138a is fixed relative to a distal-most one of the plurality of segments (e.g., 102g) such that, as wire 138a is drawn toward proximal end 18, the distal-most segment may tend to angularly displace in first direction 114, which may cause angular displacement of segment(s) proximal to the distal-most segment in the first direction. Similarly, in the depicted embodiment, wire 138b is fixed relative to the distal-most segment such that, as wire 138b is drawn toward proximal end 18, the distal-most segment may tend to angularly displace in second direction 120, which may cause angular displacement of segment(s) proximal to the distal-most segment in the second direction. In at least this way, one or more wires (e.g., 138a and/or 138b) can be configured to actuate actuatable portion 78 (e.g., between the first position and the second position) to angularly displace tip 26a relative to shaft 50. In this embodiment, body 14 includes one or more interior passageways 142 (e.g., within penetrator 70, through shaft 50 and segment(s) of actuatable portion 78), each configured to receive at least one of the one or more wires.

In the depicted embodiment, wires 138 and 138b, while being disposed on opposing sides of a pivoting axis 144 of the distal-most segment, are each disposed at an equal or substantially equal lateral distance 146 from the pivoting axis. In at least this way, as the plurality of segments rotate relative to one another in first direction 114, wire 138a may be drawn toward proximal end 18 an equal or substantially equal distance as wire 138b is drawn toward distal end 22, and, as the plurality of segments rotate relative to one another in second direction 120, wire 138b may be drawn toward the proximal end an equal or substantially equal distance as wire 138a is drawn toward the distal end (e.g., facilitating tension to be carried by each of wires 138a and 138b when actuatable portion 78 is in any position, which may promote rigidity of the actuatable portion).

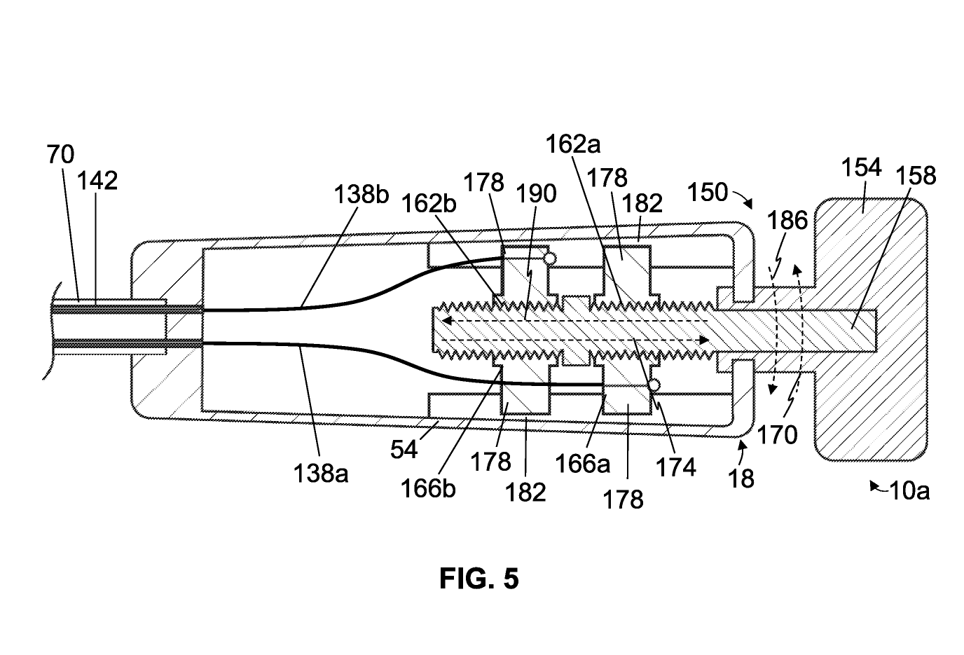

Actuatable portions (e.g., 78) of the present line passing devices may be operated or controlled in any suitable fashion. Referring now to FIG. 5, provided by way of example is an exemplary actuation mechanism 150. In the embodiment shown, actuation mechanism 150 includes a user-operable adjustment member 154 movably coupled to proximal end 18 of body 14. For example, in this embodiment, adjustment member 154 comprises a knob rotatably coupled to handle 54, which may be supported relative to the handle by one or more bearings, bushings, and/or the like.

In the depicted embodiment, adjustment member 154 is coupled to at least one of the one or more wires (e.g., 138a and/or 138b) such that movement of the adjustment member moves the wire(s) to actuate actuatable portion 78. For example, in the embodiment shown, actuation mechanism 150 includes a shaft 158 having a threaded portion 162a and/or a threaded portion 162b, where the shaft is coupled to adjustment member 154 such that rotation of the adjustment member relative to body 14 rotates at least one of the threaded portion(s) relative the body. In this embodiment, actuation mechanism 150 comprises a carrier member 166a that is (e.g., fixedly) coupled to at least one of the one or more wires (e.g., 138a) and threadably received on threaded portion 162a of shaft 158. In the depicted embodiment, carrier member 166a is restrained from rotating with shaft 158. For example, in the embodiment shown, carrier member 166a includes one or more wings 178, which may each engage a sidewall, groove, or slot 182 of handle 54 to restrain the carrier member from rotating with shaft 158. Thus, due to the threaded engagement of carrier member 166a with threaded portion 162a of shaft 158, rotation of the threaded portion relative to body 14 may cause translation of the carrier member (e.g., and any wire(s) attached to the carrier member) relative to the body. By way of illustration, in the embodiment shown, rotation of threaded portion 162a relative to body 14 in direction 170 may cause translation of carrier member 166a relative to the body in a proximal direction 174, and rotation of the threaded portion relative to the body in direction 186 may cause translation of the carrier member relative to the body in a distal direction 190. In this embodiment, at least because carrier member 166a is coupled to wire 138a, proximal translation of the carrier member relative to body 14 can cause tip 26a to angularly displace relative to shaft 50 in first direction 114, and distal translation of the carrier member relative to the body can cause (or allow) the tip to angularly displace relative to the shaft in second direction 120.

In the depicted embodiment, actuation mechanism 150 comprises a carrier member 166b that is (e.g., fixedly) coupled to at least one of the one or more wires (e.g., 138b) and threadably received on threaded portion 162b of shaft 158. While the structure and operation of carrier member 166b and threaded portion 162b may otherwise be the same as or similar to the structure and operation of carrier member 166a and threaded portion 162a, in the depicted embodiment, one of threaded portions 162a and 162b may comprise a right-handed thread (e.g., threaded portion 162a) and the other of the threaded portions may comprise a left-handed or reverse thread (e.g., threaded portion 162b). For example, in the embodiment shown, due to threaded portion 162b having a left-handed or reverse thread, rotation of the threaded portion relative to body 14 in direction 170 may cause translation of carrier member 166b relative to the body in distal direction 190, and rotation of the threaded portion relative to the body in direction 186 may cause translation of the carrier member relative to the body in proximal direction 174. In this embodiment, at least because carrier member 166b is coupled to wire 138b, proximal translation of the carrier member relative to body 14 can cause tip 26a to angularly displace relative to shaft 50 in second direction 120, and distal translation of the carrier member relative to the body can cause (or allow) the tip to angularly displace relative to the shaft in first direction 114. In these ways and others, actuation mechanism 150 may facilitate tension to be carried by each of wires 138a and 138b when actuatable portion 78 is in any position, which may promote rigidity of the actuatable portion.

In this embodiment, threaded portions 162a and 162b have a same or a similar pitch such that carrier members 166a and 166b may move a same or a similar distance relative to shaft 158 in response to rotation of the shaft (e.g., which may cooperate with wires 138a and 138b being fixed to the distal-most segment at an equal or substantially equal lateral distance 146 from a pivoting axis of the distal most segment). However, in other embodiments, a pitch of a first threaded portion (e.g., 162a) may differ from a pitch of a second threaded portion (e.g., 162b), and the pitches of the first and second threaded portions may be selected to cooperate with the relative placement of wire(s) within an actuatable portion (e.g., 78).

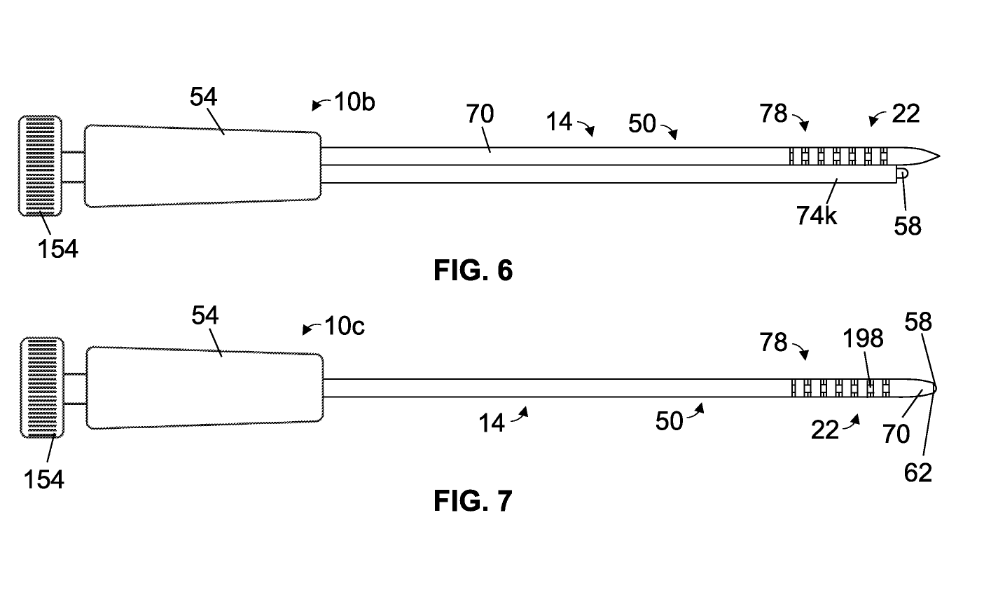

Referring now to FIG. 6, shown therein and designated by the reference numeral 10b is a second embodiment of the present line passing devices. Device 10b is substantially similar to device 10a, with the primary exception that the one or more line retainers of device 10b includes or consists of an elongated cannula 74k, which is coupled to and disposed alongside penetrator 70 from handle 54 through at least a portion of actuatable portion 78 of body 14. In the embodiment shown, elongated cannula 74k may be flexible along its entire length or rigid at shaft 50 and flexible at actuatable portion 78.

Referring now to FIG. 7, shown therein and designated by the reference numeral 10c is a third embodiment of the present line passing devices. Device 10c is substantially similar to device 10a, with the primary exception that device 10c includes a line passageway 62 that is defined within penetrator 70. For example, in the embodiment shown, a majority of (e.g., up to and including all of) line passageway 62 is defined within an outermost surface of penetrator 70. For further example, in the embodiment shown, line passageway 62 extends within penetrator 70 through shaft 50, the plurality of segments of actuatable portion 78 (e.g., within a lumen 198, which may be defined by or included in lieu of a flexible shaft 122), and the tip of the penetrator.

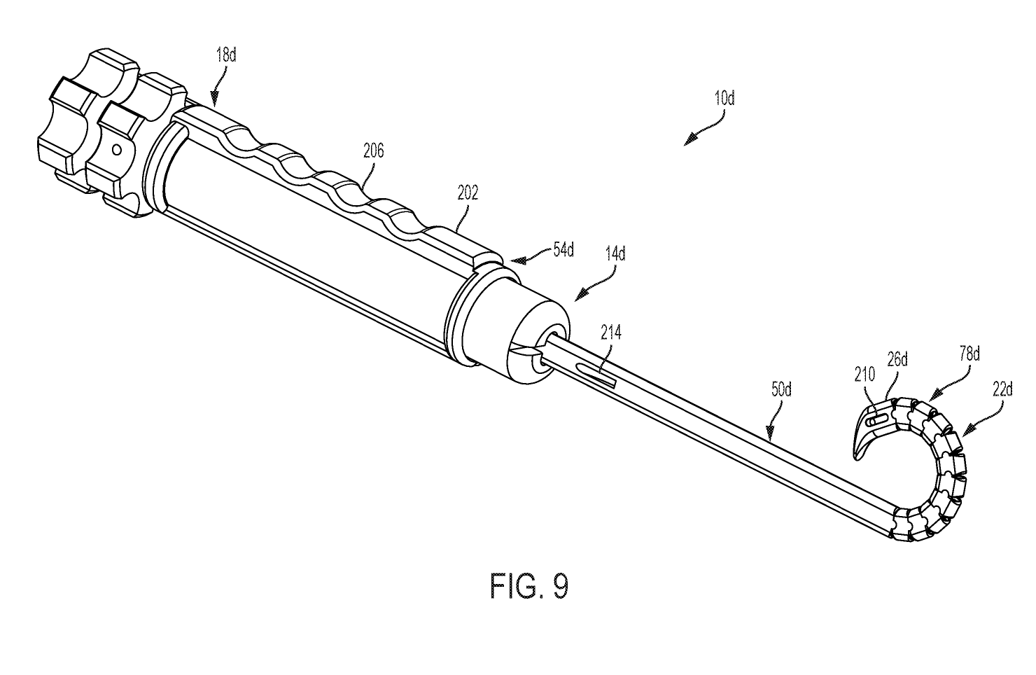

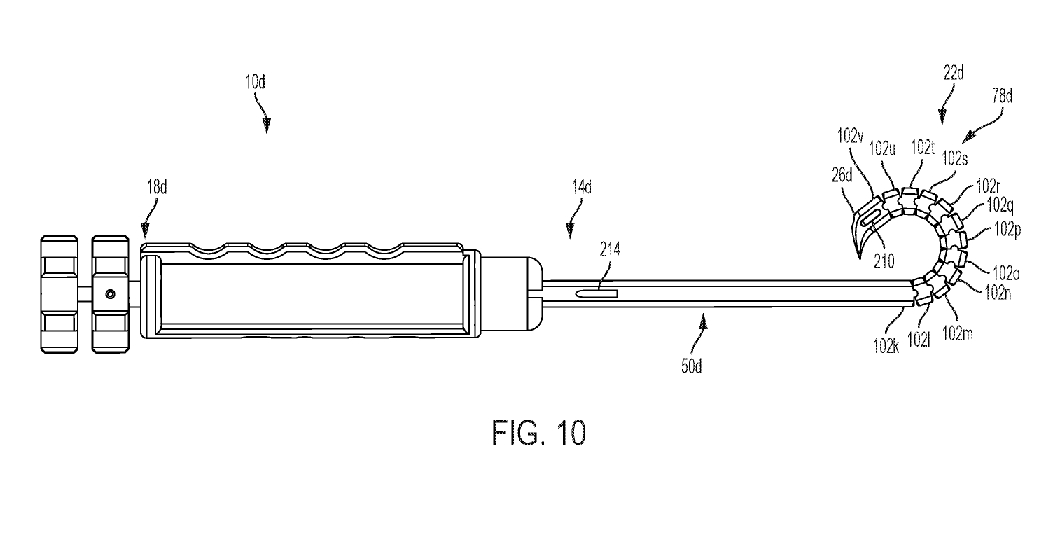

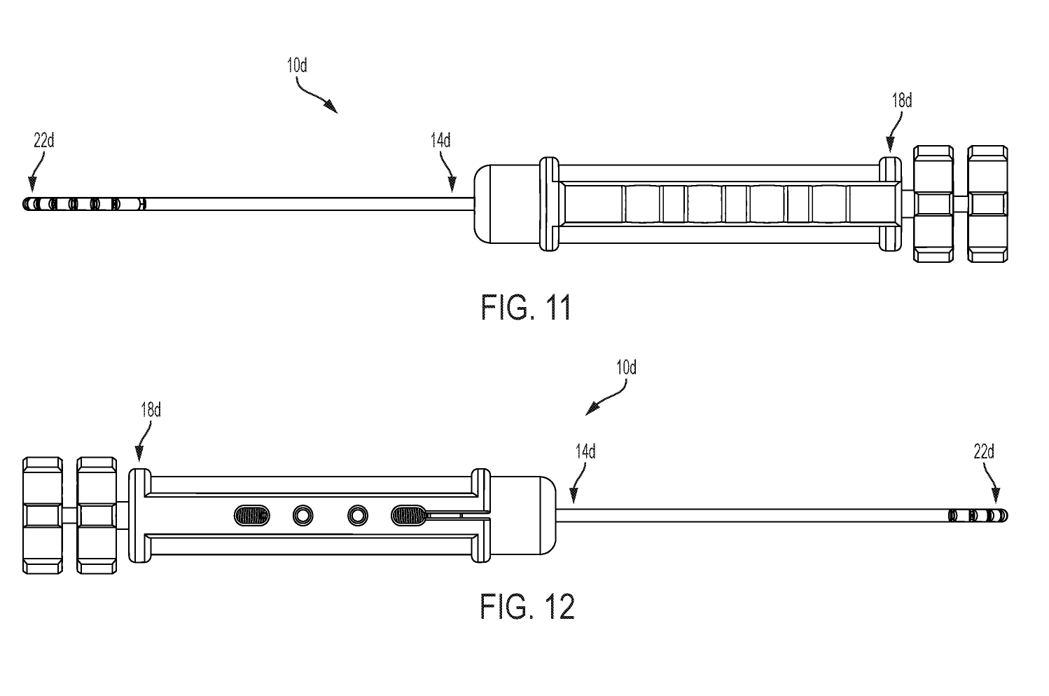

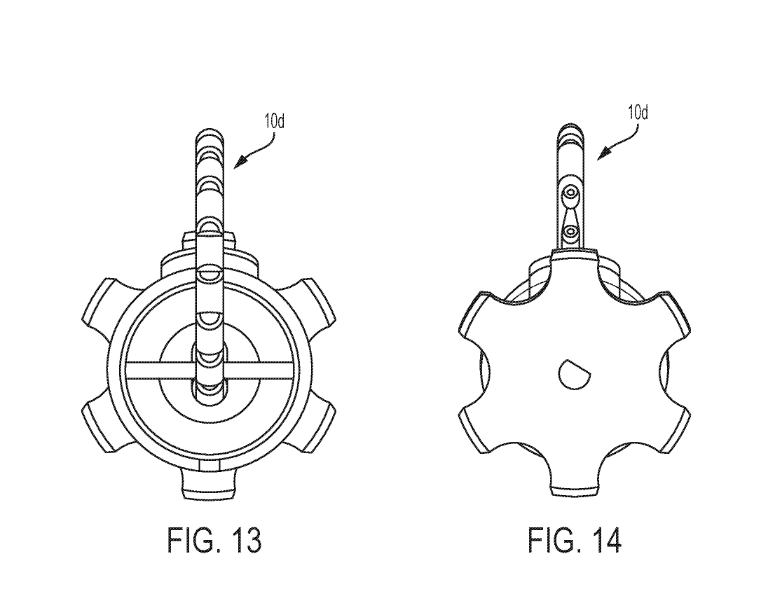

Referring now to FIGS. 9-16, shown therein and designated by the reference numeral 10d is a fourth embodiment of the present line passing devices. Device 10d can be substantially similar to device 10c, with the primary exceptions described blow. Similarly to device 10c, device 10d can include an elongated body 14d extending between a proximal end 18d and a distal end 22d, the elongated body including a handle 54d, a shaft 50d coupled to and distal to the handle, an actuatable portion 78d coupled to and distal to the shaft, and a tip 26d coupled to and distal to the actuatable portion.

Device 10d can be configured to assist a clinician in determining a position of distal end 22d of body 14d relative to a patient's tissue during use of the device, which may be particularly beneficial in instances where visibility of the distal end is obstructed (e.g., by the patient's tissue, blood, and/or the like). For example, in the embodiment shown, tip 26d can include a magnetic material, such as, for example, a rare-earth magnet (e.g., neodymium, samarium-cobalt, and/or the like), ferrite, alnico, iron, cobalt, nickel, and/or the like. In this way, for example, the clinician can determine the position of distal end 22d relative to the patient's tissue by inserting a tool, such as, for example, a probe, that is attracted to the magnetic material of tip 26d.

In this embodiment, handle 54d can include a grip 202 configured to mitigate inadvertent slippage of the handle within a clinician's hand during use of device 10d. For example, in the depicted embodiment, handle 202 defines one or more recesses or grooves 206, which can each be sized to receive a clinician's finger when the handle is disposed within the clinician's hand.

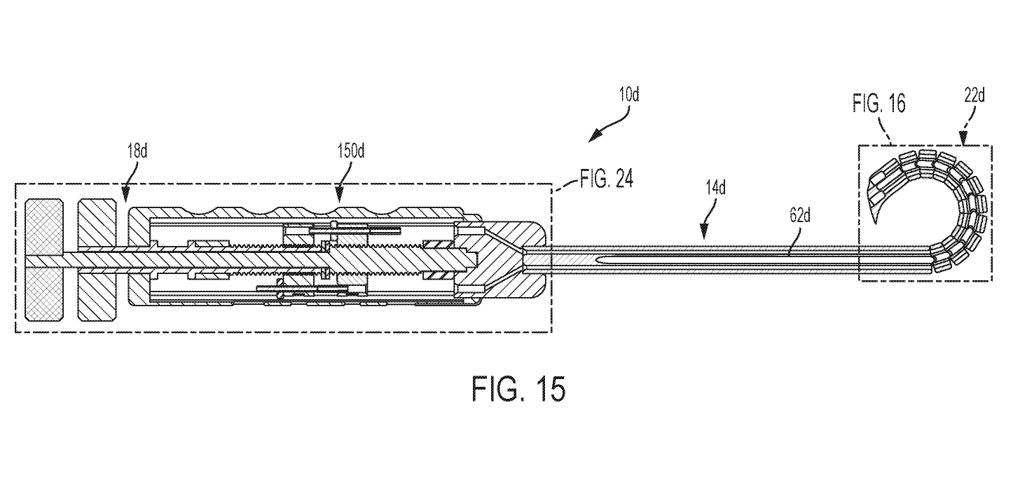

As with body 14c of device 10c, body 14d of device 10d includes a line passageway 62d (FIG. 15) extending in a direction from proximal end 18d to distal end 22d, where the line passageway extends through the distal end. More particularly, in the embodiment shown, line passageway 62d extends within and through at least a portion of each of shaft 50d, actuatable portion 78d, and tip 26d of body 14d. In this embodiment, shaft 50d defines an opening 214 in communication with line passageway 62d. In the depicted embodiment, opening 214 is defined through a side of shaft 50d, which can facilitate insertion of a line (e.g., 58) into line passageway 62d via the opening. In the embodiment shown, line passageway 62d extends through distal end 22d via an opening 210 defined by tip 26d. In this embodiment, opening 210 is defined through a side of tip 26d, which can facilitate access to a line (e.g., 58) extending from line passageway 62d, which might otherwise be obstructed by a patient's tissue at the distal-most portion of the tip, mitigate clogging of the opening with a patient's tissue, and/or the like.

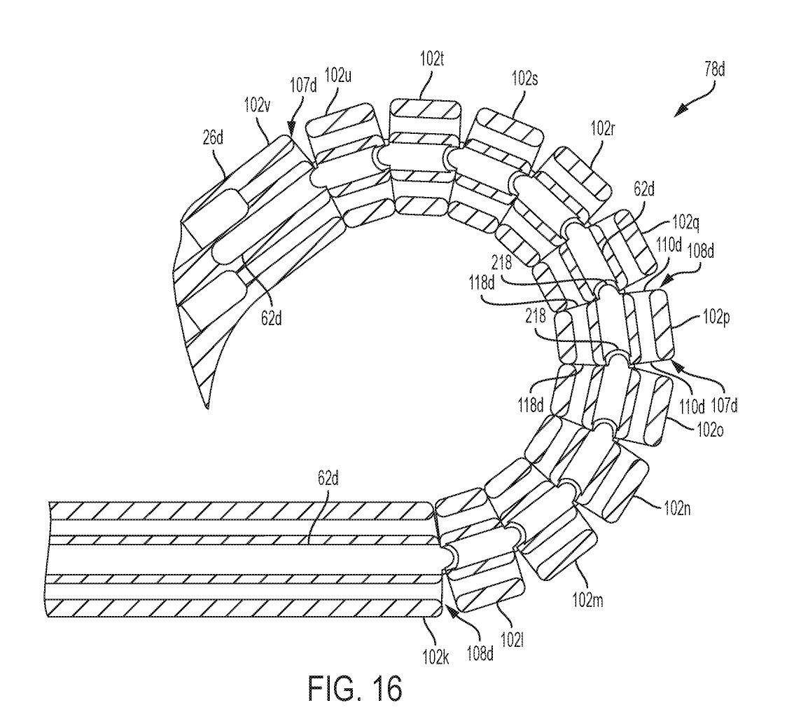

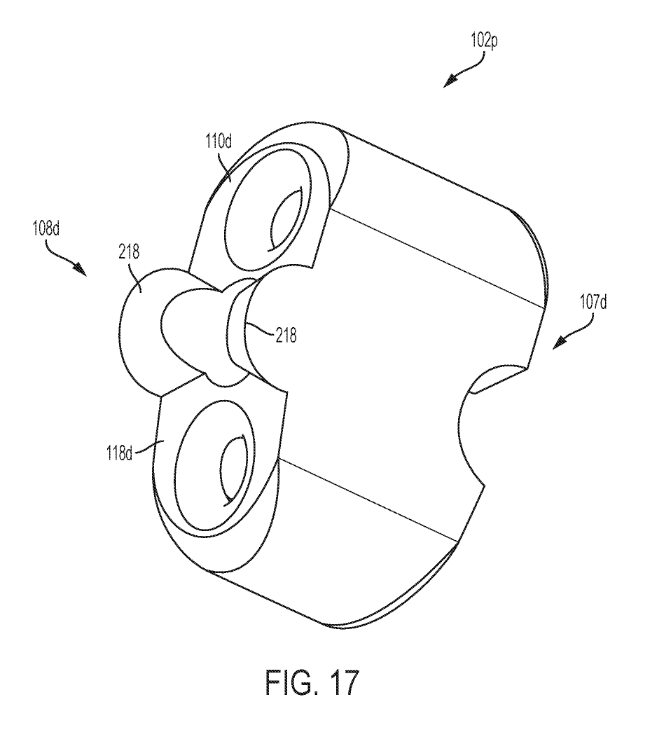

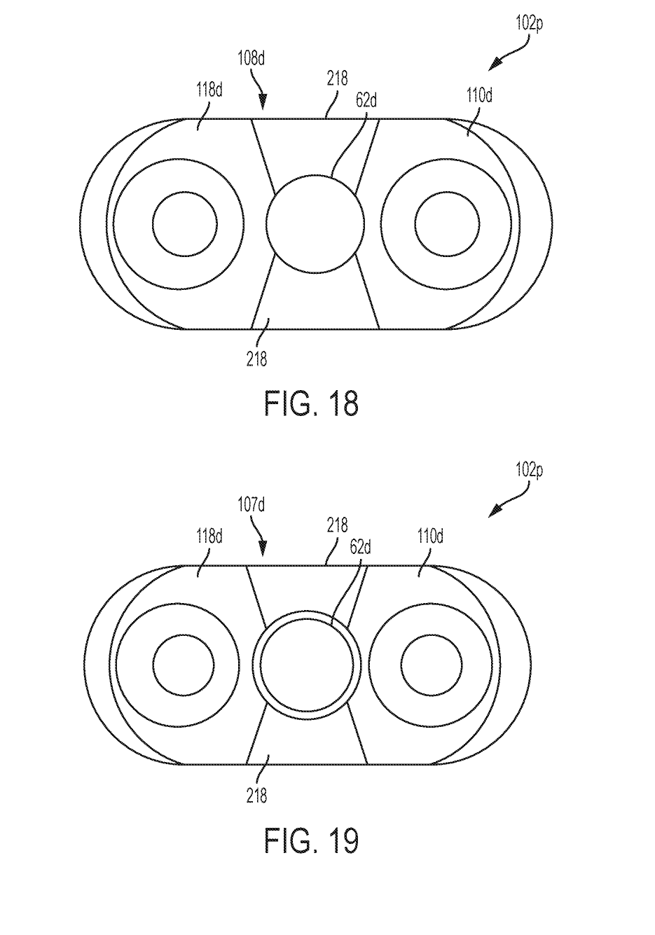

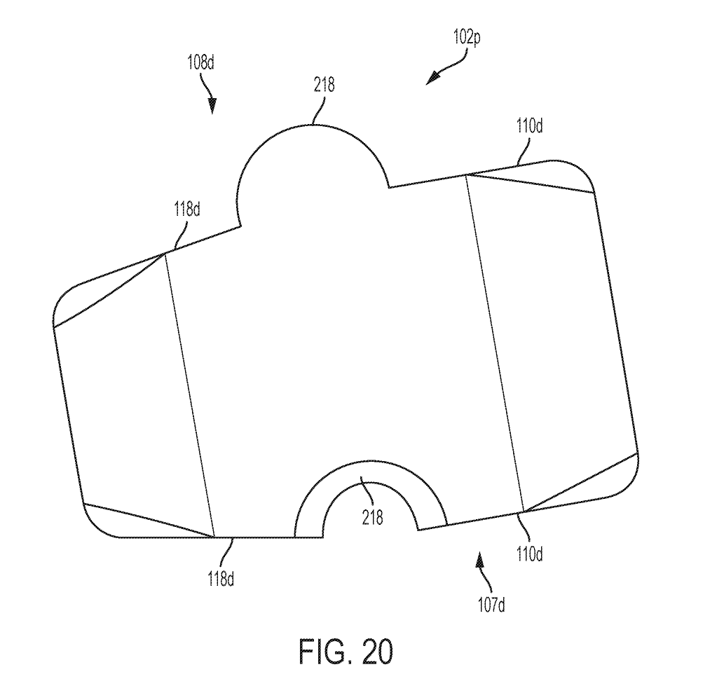

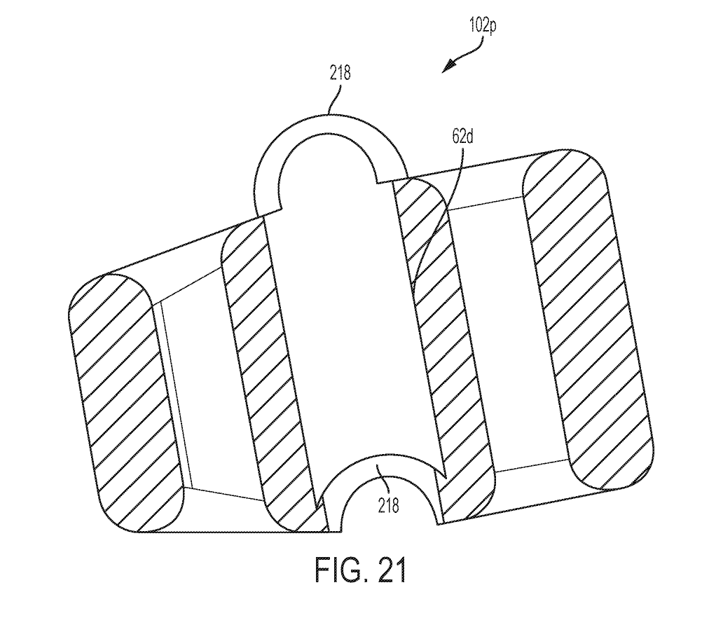

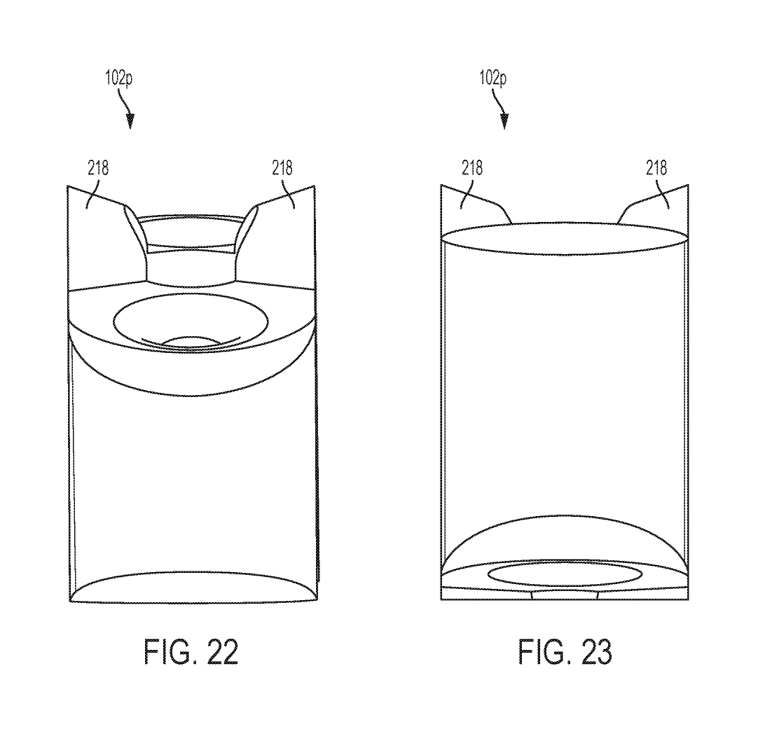

Similarly to device 10c, actuatable portion 78d of device 10d includes a plurality of segments (e.g., 102k-102v), each having a proximal face 107d and/or a distal face 108d that defines a first mating surface 110d and a second mating surface 118d (FIG. 16). Referring additionally to FIGS. 17-23, in the depicted embodiment, for each of the segments, at least one of proximal face 107d and distal face 108d includes an arcuate bearing surface 218 disposed between first mating surface 110d and second mating surface 118d, the arcuate bearing surface configured to contact a corresponding arcuate bearing surface 218 of an adjacent one of the segments. To illustrate, for concave arcuate bearing surface(s) 218, corresponding arcuate bearing surface(s) 218 are convex, and for convex arcuate bearing surface(s) 218, corresponding arcuate bearing surface(s) are concave. In the embodiment shown, arcuate bearing surfaces 218 are conical; however, in other embodiments, segments (e.g., 102k-102v) can include arcuate bearing surfaces (e.g., 218) that are cylindrical. Such arcuate bearing surfaces 218 can facilitate relative positioning of the segments, enhance the rigidity of actuatable portion 78d, and/or the like.

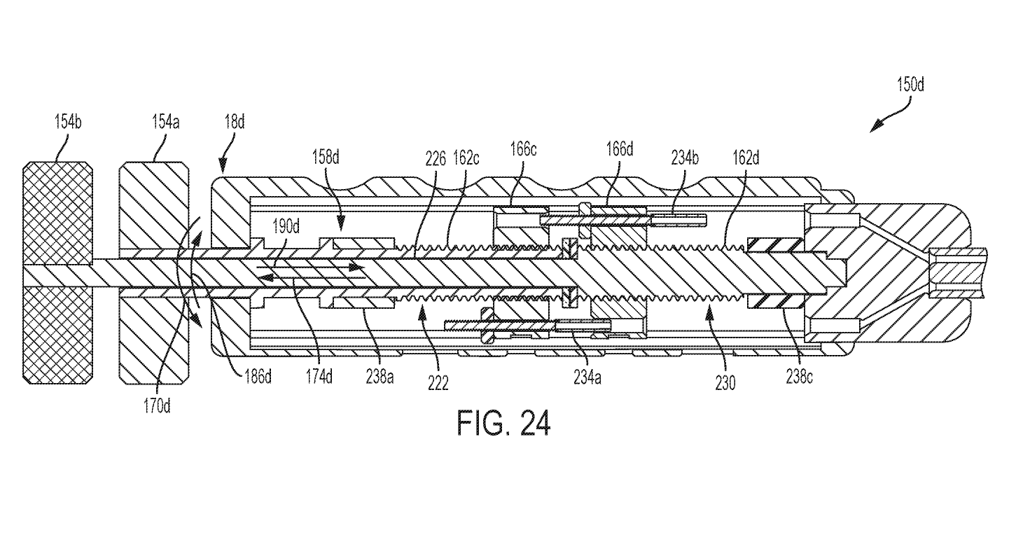

Referring now to FIG. 24, provided by way of example is an exemplary actuation mechanism 150d. As with actuation mechanism 150, actuation mechanism 150d includes a shaft 158d having a threaded portion 162c and a threaded portion 162d, with a carrier member 166c threadably received on threaded portion 162c and a carrier member 166d threadably received on threaded portion 162d. In the embodiment shown, wires (e.g., 138a and 138b) can be coupled to carrier members 166c and 166d such that a tension in the wire(s) is adjustable. For example, in this embodiment, actuation mechanism 150 can comprise wire retention assemblies, 234a and 234b, each configured to couple a wire to a carrier member (e.g., wire 138a to carrier member 166c and wire 138b to carrier member 166d, respectively). In the depicted embodiment, wire retention assemblies 234a and 234b each include a threaded rod and a fitting coupled to the threaded rod, the fitting configured to be coupled to a wire (e.g., 138a or 138b), where the threaded rod can be received through an opening of a carrier member (e.g., 166c or 166d) and retained relative to the carrier member via a threaded fastener. In this way, a tension in the wire can be adjusted by varying a position of the threaded fastener relative to the threaded rod.

Similarly to actuation mechanism 150, in actuation mechanism 150d, rotation of threaded portion 162c relative to body 14d in direction 170d may cause translation of carrier member 166c relative to the body in a proximal direction 174d (e.g., and, by tensioning wire 138a, angular displacement of tip 26d relative to shaft 50d in first direction 114), and rotation of the threaded portion relative to the body in direction 186d may cause translation of the carrier member relative to the body in a distal direction 190d (e.g., and, by relaxing wire 138a, allowing angular displacement of tip 26d relative to shaft 50d in second direction 120). Similarly, rotation of threaded portion 162d relative to body 14d in direction 170d may cause translation of carrier member 166d relative to the body in distal direction 190d (e.g., and, by relaxing wire 138b, allowing angular displacement of tip 26d relative to shaft 50d in first direction 114), and rotation of the threaded portion relative to the body in direction 186d may cause translation of the carrier member relative to the body in proximal direction 174d (e.g., and, by tensioning wire 138b, angular displacement of tip 26d relative to shaft 50d in second direction 120). As shown, actuation mechanism 150d includes one or more stops (e.g., 238a and/or 238c) configured to limit translation of carrier member 166c and/or carrier member 166d relative to body 14d.

In the embodiment shown, threaded portion 162c is rotatable relative to body 14d independently of rotation of threaded portion 162d relative to the body. For example, threaded portion 162c can be rotated relative to body 14d without rotating threaded portion 162d relative to the body, and threaded portion 162d can be rotated relative to the body without rotating threaded portion 162c relative to the body. In this embodiment, shaft 158d includes an outer spindle 222 that defines an interior channel 226 and an inner spindle 230 that is rotatably disposed within and extends from the interior channel. In the depicted embodiment, threaded portion 162c can be defined by outer spindle 222, and threaded portion 162d can be defined by a portion of inner spindle 230 that is disposed outside of interior channel 226 of the outer spindle.

In the embodiment shown, actuation mechanism 150d includes a user-operable adjustment member 154a and a user-operable adjustment member 154b. As shown, adjustment member 154a may be coupled to shaft 158d (e.g., outer spindle 222 thereof) such that rotation of the adjustment member relative to body 14d rotates threaded portion 162c relative to the body. Similarly, in this embodiment, adjustment member 154b may be coupled to shaft 158d (e.g., inner spindle 230 thereof) such that rotation of the adjustment member relative to body 14d rotates threaded portion 162d relative to the body. In at least this way, actuation mechanism 150d can allow a clinician to adjust a position of actuatable portion 78d (e.g., by rotating adjustment members 154a and 154b relative to body 14d in unison) as well as adjust a rigidity of the actuatable portion (e.g., by rotating the adjustment members relative to one another).

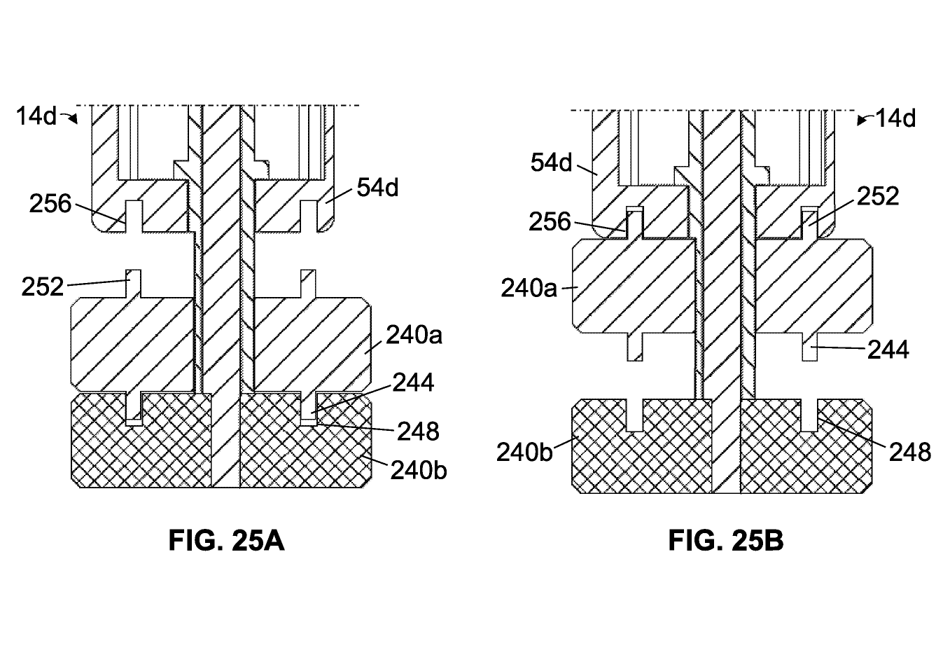

Referring now to FIGS. 25A and 25B, shown are knobs, 240a and 240b, that may be suitable for use in some embodiments (e.g., 10a, 10b, 10c, 10d, 10e, and/or the like) of the present line passing devices (e.g., as adjustment members 154a and 154b of device 10d). In the embodiment shown, knob 240a is movable relative to knob 240b between a first position (FIG. 25A) in which knob 240a is engaged with knob 240b such that rotation of knob 240b relative to body 14d rotates knob 240a relative to the body, and a second position (FIG. 25B) in which knob 240a is disengaged with knob 240b such that knob 240b is rotatable relative to the body independently of rotation of knob 240a relative to the body. For example, in this embodiment, knob 240a is slidably coupled to shaft 158d. In the depicted embodiment, knob 240a includes one or more protrusions 244 and/or one or more recesses, and knob 240b includes one or more recesses 248 and/or one or more protrusions, each configured to receive a corresponding one of the protrusion(s) of or be received by a corresponding one of the recess(es) of knob 240a when knob 240a is in the first position.

In the depicted embodiment, knob 240a is movable to a position (e.g., FIG. 25B) in which the knob is restrained from rotation relative to body 14d. For example, knob 240a can include one or more protrusions 252 and/or one or more recesses, and body 14d (e.g., handle 54d thereof) can include one or more recesses 256 and/or one or more protrusions, each configured to receive a corresponding one of the protrusion(s) of or be received by a corresponding one of the recess(es) of knob 240a.

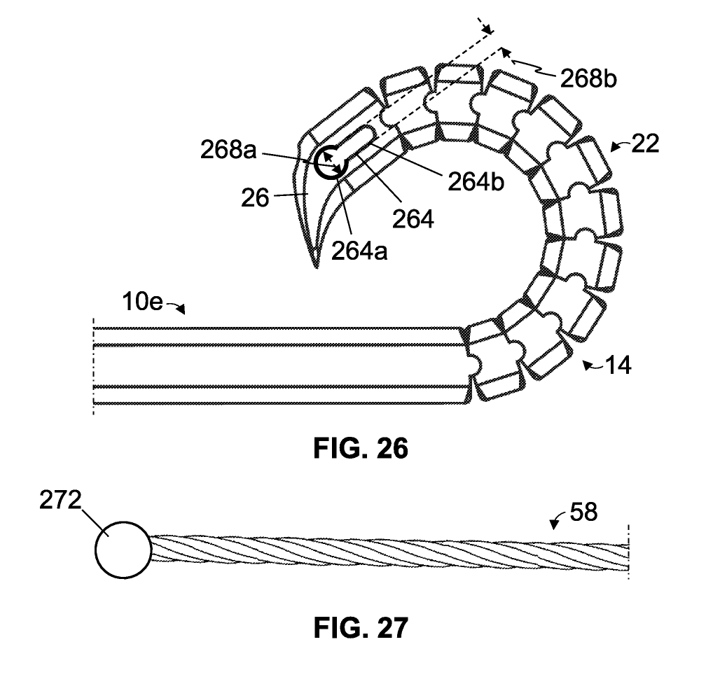

Referring now to FIG. 26, shown is a fifth embodiment 10e of the present line passing devices. Device 10e may be substantially similar to device 10d, with the primary exceptions described below. In the embodiment shown, distal end 22 of body 14 is configured to be coupled to a line (e.g., 58) such that, for example, a majority of the line is disposed outside of the body. For example, in this embodiment, distal end 22 of body 14 (e.g., tip 26 thereof) defines an opening 264 configured to receive the line. More particularly, in the depicted embodiment, opening 264 can include a first portion 264a having a first width 268a and a second portion 264b in communication with the first portion, the second portion having a second width 268b that is smaller than the first width. As shown in FIG. 27, line 58 can include a fitting 272 coupled to an end of the line, where the line can be coupled to distal end 22 by inserting the fitting through first portion 264a of opening 264 and moving the line relative to the opening such that the line is disposed within second portion 264b of the opening. To illustrate, during use, a clinician can maneuver device 10e into position around a bone of a patient, after which the clinician can couple line 58 to distal end 22. Once line 58 is coupled to distal end 22, the clinician can withdraw the device to pull the line around the bone, which can be facilitated by relaxing actuatable portion 78 (e.g., by rotating adjustment members 154a and 154b relative to one another, if the device includes actuation mechanism 150d or a similar actuation mechanism). Such devices (e.g., 10e) may not include a line passageway (e.g., 26) that extends within and/or through shaft 50, actuatable portion 78, and/or tip 26.

Embodiments of the present devices can be configured for a variety of use(s), whether those use(s) are in addition to or in lieu of line passing. For example, some embodiments of the present devices can be configured to perform electrocautery (e.g., by including a heatable tip 26 or a heatable element, such as a needle or a probe, coupled to and/or extendable from the tip), biopsies (e.g., in which a tissue sample can be received by a tip 26), scoping procedures (e.g., by including a camera coupled to and/or extendable from a tip), suctioning procedures (e.g., by including a vacuum source in fluid communication with a passageway that extends through a tip 26), and/or the like. For further example, some embodiments of the present devices can be configured for deploying devices other than lines (e.g., 58), such as, for example, implants, drugs, tracking devices, or other deployable devices (e.g., and such a device can include a passageway within which a deployable device is disposed and from which the deployable device can be deployed). For yet further example, some embodiments of the present devices can include light sources (e.g., coupled to a tip 26), guidance technology (e.g., marker(s), transducer(s), and/or the like disposed on a device and configured to facilitate positioning of the device relative to a patient's tissue), and/or the like. For yet further example, some devices can be configured to be directed through soft tissue or down a path defined by tissue (e.g., a tendon sheath, lumen organ, and/or the like). For yet further example, the present devices can have any suitable dimensions (e.g., for handle 54, shaft 50, actuatable portion 78, tip 26, and/or the like), and such dimensions can be selected depending on, for example, the intended use of the devices.

Some embodiments of the present devices may be included in some embodiments of the present kits. Some of the present kits may include a container (e.g., a tray, such as a sealed tray, a box, or a pouch, such as a sealed and/or flexible pouch) in which component(s) (e.g., device 10a, 10b, 10c, 10d, 10e, and/or the like, line(s) 58, tunneler 194, and/or the like) are disposed. Some of the present kits may include instructions for use disposed, for example, on the outside of the container (e.g., on a sticker) or on material disposed inside the container (e.g., on a written insert). In some of the present kits, one or more of the component(s) may be sterile.

For example, some of the present kits include a line passing device (e.g., 10a, 10b, 10c, 10d, and/or the like) including an elongated body (e.g., 14, 14d, and/or the like) having a proximal end (e.g., 18, 18d, and/or the like), a distal end (e.g., 22, 22d, and/or the like) including a tip (e.g., 26a, 26b, 26c, 26d, and/or the like) and an actuatable portion (e.g., 78, 78d, and/or the like) proximal to the tip, and a line passageway (e.g., 62, 62d, and/or the like) sized to receive a line (e.g., 58) and extending in a direction from the proximal end to the distal end, the line passageway extending through the distal end, a user-operable adjustment member (e.g., 154, 154a, 154b, and/or the like) movably coupled to the proximal end, and one or more wires (e.g., 138a, 138b, and/or the like) coupled to the adjustment member and to the actuatable portion, where the line passing device is configured such that, in response to movement of the adjustment member, the one or more wires actuate the actuatable portion to angularly displace the distal end relative to the proximal end (e.g., to or to a position between the position depicted in FIG. 3A and/or the position depicted in FIG. 3B). In some of the present kits, the line passing device is configured such that, in response to movement of the adjustment member, the one or more wires actuate the actuatable portion between a first rigid position in which the distal end is axially aligned with the proximal end (e.g., FIG. 3A) and a second rigid position in which the distal end is angularly displaced relative to the proximal end (e.g., FIG. 4B).

In some of the present kits, the body includes a penetrator (e.g., 70) that includes the tip and one or more line retainers (e.g., 74a-74h) coupled to and disposed alongside the penetrator, the one or more line retainers defining at least a portion of the line passageway. Some of the present kits include a cerclage cable sized to be received within the line passageway. In some of the present kits, the cerclage cable is at least partially disposed within the line passageway. Some of the present kits include a suture sized to be received within the line passageway. In some of the present kits, the suture is at least partially disposed within the line passageway. Some of the present kits include a tunneler (e.g., 194).

Some embodiments of the present methods for performing a cerclage comprise inserting a distal end (e.g., 22, 22d, or the like) of an elongated body (e.g., 14, 14d, or the like) into an insertion site on a patient, the body including a proximal end (e.g., 18, 18d, or the like) and a line passageway (e.g., 62, 62d, or the like) extending in a direction from the proximal end to the distal end, where the distal end comprises an actuatable portion (e.g., 78, 78d, or the like), actuating the actuatable portion to angularly displace the distal end relative to the proximal end (e.g., to or toward the position depicted in FIG. 3B) such that at least a portion of the body at least partially surrounds a bone of the patient, and passing a cerclage cable out of the line passageway and at least partially around the bone.

Some of the present methods comprise inserting a tunneler (e.g., 194) into the insertion site on the patient. Some of the present methods comprise tensioning the cerclage cable. Some of the present methods comprise actuating the actuatable portion to angularly displace the distal end relative to the proximal end toward a position in which the distal end is axially aligned with the proximal end (e.g., to or toward the position depicted in FIG. 3A) and removing the body from the insertion site.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A line passing device comprising:
an elongated body extending between a proximal end and a distal end, the elongated body including:
a shaft;
an actuatable portion coupled to and distal to the shaft;
a tip coupled to and distal to the actuatable portion; and
a line passageway sized to receive a line and extending in a direction from the proximal end to the distal end, the line passageway extending through the distal end; and
an actuation mechanism comprising:
first and second carrier members, each carrier member coupled to the body such that the carrier member is translatable relative to the body;
one or more wires coupled between the first carrier member and the actuatable portion such that proximal translation of the first carrier member relative to the body actuates the actuatable portion to angularly displace the tip relative to the shaft in a first direction; and
one or more wires coupled between the second carrier member and the actuatable portion such that proximal translation of the second carrier member relative to the body actuates the actuatable portion to angularly displace the tip relative to the shaft in a second direction that is opposite to the first direction.

2. The line passing device of claim 1, wherein:
the actuation mechanism comprises a shaft coupled to the body, the shaft having first and second threaded portions, each threaded portion being rotatable relative to the body;
the first carrier member is threadably coupled to the first threaded portion such that rotation of the first threaded portion relative to the body translates the first carrier member relative to the body; and
the second carrier member is threadably coupled to the second threaded portion such that rotation of the second threaded portion relative to the body translates the second carrier member relative to the body.

3. The line passing device of claim 2, wherein the actuation mechanism comprises a knob coupled to the shaft of the actuation mechanism such that rotation of the knob relative to the body rotates at least one of the first threaded portion and the second threaded portion relative to the body.

4. The line passing device of claim 2, wherein the first threaded portion is rotatable relative to the body independently of rotation of the second threaded portion relative to the body.

5. The line passing device of claim 2, wherein the actuation mechanism comprises:
a first knob coupled to the shaft of the actuation mechanism such that rotation of the first knob relative to the body rotates the first threaded portion relative to the body; and
a second knob coupled to the shaft of the actuation mechanism such that rotation of the second knob relative to the body rotates the second threaded portion relative to the body.

6. The line passing device of claim 5, wherein the first knob is movable relative to the second knob between:
a first position in which the first knob is engaged with the second knob such that rotation of the second knob relative to the body rotates the first knob relative to the body; and
a second position in which the first knob is disengaged with the second knob such that the second knob is rotatable relative to the body independently of rotation of the first knob relative to the body.

7. The line passing device of claim 2, wherein the shaft of the actuation mechanism includes:
an outer spindle defining an interior channel, the outer spindle including the first threaded portion; and
an inner spindle rotatably disposed within and extending from the interior channel of the outer spindle, wherein a portion of the inner spindle that is disposed outside of the interior channel includes the second threaded portion.

8. The line passing device of any of claims 1-7, wherein:
the actuatable portion includes a plurality of segments, each having a proximal face and/or a distal face that defines a first mating surface and a second mating surface;
for at least one of the faces of at least one of the segments, the second mating surface is angularly disposed relative to the first mating surface; and
the actuatable portion is actuatable between:
a first position in which adjacent ones of the segments are in contact with one another along their first mating surfaces; and
a second position in which adjacent ones of the segments are in contact with one another along their second mating surfaces.

9. The line passing device of claim 8, wherein:
when the actuatable portion is in the first position, the tip is axially aligned with the shaft of the body; and
when the actuatable portion is in the second position, the tip is not axially aligned with the shaft of the body.

10. A line passing device comprising:
an elongated body extending between a proximal end and a distal end, the elongated body including:
a shaft;
an actuatable portion coupled to and distal to the shaft, the actuatable portion including:
a plurality of segments, each having a proximal face and/or a distal face that defines a first mating surface and a second mating surface;
wherein, for at least one of the faces of at least one of the segments, the second mating surface is angularly disposed relative to the first mating surface;
a tip coupled to and distal to the actuatable portion; and
a line passageway sized to receive a line and extending in a direction from the proximal end to the distal end, the line passageway extending through the distal end; and
an actuation mechanism comprising one or more wires coupled to the actuatable portion, the one or more wires configured to angularly displace the tip relative to the shaft by actuating the actuatable portion between:
a first position in which adjacent ones of the segments are in contact with one another along their first mating surfaces; and
a second position in which adjacent ones of the segments are in contact with one another along their second mating surfaces.

11. The line passing device of claim 10, wherein:
when the actuatable portion is in the first position, adjacent ones of the segments are not in contact with one another along their second mating surfaces; and
when the actuatable portion is in the second position, adjacent ones of the segments are not in contact with one another along their first mating surfaces.

12. The line passing device of claim 10, wherein:
when the actuatable portion is in the first position, the tip is axially aligned with the shaft; and
when the actuatable portion is in the second position, the tip is not axially aligned with the shaft.

13. The line passing device of claim 10, wherein, for each of the segments:
at least one of the faces includes an arcuate bearing surface disposed between the first mating surface and the second mating surface; and
the arcuate bearing surface is configured to contact a corresponding arcuate bearing surface of an adjacent one of the segments as the actuatable portion is actuated between the first position and the second position.

14. The line passing device of claim 13, wherein, for at least one of the segments, the arcuate bearing surface(s) are conical.

15. The line passing device of any of claims 10-14, wherein:
the actuation mechanism comprises a first carrier member coupled to the body such that the first carrier member is translatable relative to the body; and
at least one of the one or more wires is coupled between the first carrier member and the actuatable portion such that translation of the first carrier member relative to the body actuates the actuatable portion to angularly displace the tip relative to the shaft.

16. The line passing device of claim 15, wherein:
the actuation mechanism comprises a second carrier member coupled to the body such that the second carrier member is translatable relative to the body; and
at least one of the one or more wires is coupled between the second carrier member and the actuatable portion such that translation of the second carrier member relative to the body actuates the actuatable portion to angularly displace the tip relative to the shaft.

17. The line passing device of claim 16, wherein:
proximal translation of the first carrier member relative to the body angularly displaces the tip relative to the shaft in a first direction; and
proximal translation of the second carrier member relative to the body angularly displaces the tip relative to the shaft in a second direction that is opposite to the first direction.

18. The line passing device of any of claim 1-7 or 10-14, wherein the tip defines an opening in communication with the line passageway, the opening being defined through a side of the tip.

19. The line passing device of any of claim 1-7 or 10-14, wherein the shaft of the body defines an opening in communication with the line passageway, the opening being defined through a side of the shaft.

20. The line passing device of any of claim 1-7 or 10-14, wherein the body includes a handle coupled to and proximal to the shaft of the body.

21. A method for performing a cerclage, comprising:
inserting a distal end of an elongated body into an insertion site on a patient, the body including:
a proximal end; and
a line passageway extending in a direction from the proximal end to the distal end;
where the distal end comprises an actuatable portion;
actuating the actuatable portion to angularly displace the distal end relative to the proximal end such that at least a portion of the body at least partially surrounds a bone of the patient; and
passing a cerclage cable out of the line passageway and at least partially around the bone.

22. The method of claim 21, comprising:
actuating the actuatable portion to angularly displace the distal end relative to the proximal end toward a position in which the distal end is axially aligned with the proximal end; and
removing the body from the insertion site.

23. A line passing device comprising:
an elongated body extending between a proximal end and a distal end, the elongated body including:
a shaft;
an actuatable portion coupled to and distal to the shaft; and
a tip coupled to and distal to the actuatable portion;
wherein the distal end of the body is configured to be coupled to a line; and
an actuation mechanism comprising:
first and second carrier members, each carrier member coupled to the body such that the carrier member is translatable relative to the body;
one or more wires coupled between the first carrier member and the actuatable portion such that proximal translation of the first carrier member relative to the body actuates the actuatable portion to angularly displace the tip relative to the shaft in a first direction; and
one or more wires coupled between the second carrier member and the actuatable portion such that proximal translation of the second carrier member relative to the body actuates the actuatable portion to angularly displace the tip relative to the shaft in a second direction that is opposite to the first direction.

24. A line passing device comprising:
an elongated body extending between a proximal end and a distal end, the elongated body including:
a shaft;
an actuatable portion coupled to and distal to the shaft, the actuatable portion including:
a plurality of segments, each having a proximal face and/or a distal face that defines a first mating surface and a second mating surface;
wherein, for at least one of the faces of at least one of the segments, the second mating surface is angularly disposed relative to the first mating surface; and
a tip coupled to and distal to the actuatable portion;
wherein the distal end of the body is configured to be coupled to a line;
an actuation mechanism comprising one or more wires coupled to the actuatable portion, the one or more wires configured to angularly displace the tip relative to the shaft by actuating the actuatable portion between:
a first position in which adjacent ones of the segments are in contact with one another along their first mating surfaces; and
a second position in which adjacent ones of the segments are in contact with one another along their second mating surfaces.

25. The line passing device of claim 23 or 24, wherein the distal end of the body defines an opening configured to receive the line.

26. The line passing device of claim 25, wherein:
the line includes a fitting coupled to an end of the line;
the opening includes a first portion having a first width and a second portion in communication with the first portion, the second portion having a second width that is smaller than the first width; and
the line is configured to be coupled to the distal end of the body by:
inserting the fitting through the first portion of the opening; and
moving the line relative to the opening such that the line is disposed within the second portion of the opening.
